(19) United States
(12) Patent Application Publication
(10) Pub. No.: US 2025/0149176 A1
(43) Pub. Date: May 8, 2025

(54) MODEL-BASED RISK PREDICTION AND TREATMENT PATHWAY PRIORITIZATION

(71) Applicant: Optum Services (Ireland) Limited, Dublin (IE)

(72) Inventors: David Alexander Dickie, Renfrewshire (GB); James McNair Sloan, Bathgate (GB); Michael J. McCarthy, Dublin (IE); Kieran O'Donoghue, Dublin (IE)

(21) Appl. No.: 18/504,638

(22) Filed: Nov. 8, 2023

(51) Int. Cl. G16H 50/30; A61B 5/00; G06T 7/00; G06T 7/70; G16H 20/00; G16H 40/20; G16H 40/67; G16H 50/20

(52) U.S. Cl. CPC G16H 50/30; A61B 5/4088; A61B 5/7275; G06T 7/0012; G06T 7/70; G16H 20/00; G16H 50/20; G16H 40/20; G16H 40/67

(57) ABSTRACT

Various embodiments of the present disclosure provide machine learning model-based risk prediction and treatment pathway prioritization for entities associated with a respective disparity group. Example embodiments are configured to generate, using a risk prediction model, an individual risk score for an entity of a disparity group associated with an entity cohort. Example embodiments are also configured to generate, using a disparity risk adjustment model, a disparity adjusted risk score for the entity based on the individual risk score. Example embodiments are also configured to initiate various prediction-based actions for the entity based on a comparison between the disparity adjusted risk score and a risk score threshold. Example embodiments are also configured to generate a phenotypic profile for the entity based on an evaluation data object and an image-based evaluation data object for the entity and generate a prediction-based action sequence for the entity based on the phenotypic profile.

### BACKGROUND

Various embodiments of the present disclosure address technical challenges related to computer-based risk predictions for individuals that have developed or may develop a particular disease, disorder, and/or impairment, such as cognitive impairment, dementia, heart disease, diabetes, renal disease, and/or the like. Limited primary care capacity has led to bottlenecks in service provision, variability in care pathways, and delayed diagnoses. Additionally, existing computer-based techniques for determining an individual risk that an entity (e.g., a medical patient) will develop a particular disease, ailment, and/or impairment are lacking in that the existing techniques are incapable of reliability detecting a high risk entity and, once identified, generating treatment pathway tailored to the high risk entity.

Traditionally, risk prediction models rely on cohort or “population-level” insights to generate predictions for an entity of the cohort, which limits the type of data and insights available to such models. For example, structural changes in an entity's body that occur years prior to disease, disorder, and/or impairment symptom onset provide useful insights for detecting risk. However, population-level evaluation and/or analysis for such insights is too computationally and resource intensive. Clinical investigations for most diseases, disorders, and/or impairments, which may be considered by traditional models, generally do not start until difficulties with symptoms impact the daily life of a respective entity. By this time, treatment outcomes are unfavorable and even new disease modifying treatments and/or techniques may be ineffective.

Traditional risk prediction techniques utilize risk profiling to prioritize population-level interventions. For example, a predetermined percentage of patients at risk for emergency medical admissions due to high blood pressure may be enrolled in a blood pressure lowering program. However, these existing approaches fail to address regional and/or demographic inequalities within a prediction domain. Furthermore, existing risk prediction approaches do not perform staged evaluations and reintegration of evaluation results back into individual risk data prior to suggesting and/or performing prediction-based decisions. As such, traditional techniques are limited to particular sequences of data that preclude and fail to holistically account for available data of different, traditionally incompatible data types (e.g., cognitive scores, electronic health record (EMR) data, image scans, remote monitoring data, etc.).

Various embodiments of the present disclosure make important contributions to traditional risk prediction techniques by addressing these technical challenges, among others.

### BRIEF SUMMARY

Various embodiments of the present disclosure disclose machine-learning (ML) techniques that improve traditional risk prediction techniques by enabling the prediction of risk that a particular entity associated with a respective disparity group, entity cohort, and/or historical entity datastore (e.g., an electronic medical record (EMR) datastore) has, or will develop a particular disease, condition, malady, sickness, disorder, and/or impairment. In some examples, a risk prediction model may include one or more machine learning models configured to generate individual risk scores, disparity adjusted risk scores, evaluation adjusted risk scores, recommendations, and/or phenotypic profiles for one or more entities associated with the respective disparity group, entity cohort, and/or historical entity datastore. Unlike traditional techniques, the risk model may leverage a staged processing scheme to account for various types of data associated with an entity cohort. For instance, the risk prediction model may integrate numerical data, such as cognitive scores in a clinical domain, textual data, such as EMR data, image data, such as image scans, and/or the like to predict and incrementally refine individual risk score for entities within an entity cohort. The risk prediction model may also be configured to initiate and/or cause execution of one or more prediction-based actions for one or more respective entities based on a comparison of one or more of a disparity adjusted risk score and/or an evaluation adjusted risk score associated with a respective entity to one or more risk score thresholds. In this regard, the risk prediction model may initiate and/or facilitate the execution of one or more entity evaluation actions, image-based entity evaluation actions, entity monitoring actions, and/or entity evaluation cessation actions. By doing so, the risk prediction model may continuously gain real time data and integrate the real time data with historical data to refine predictive scores at an entity-level within an entity cohort.

Furthermore, based on a phenotypic profile generated for a respective entity, the risk prediction model may also be configured to generate a prediction-based action sequence associated with a particular treatment plan and/or set of recommendations configured to treat, alleviate, address, mitigate, and/or otherwise manage a particular disease, condition, malady, sickness, disorder, and/or impairment associated with the respective entity. In this manner, using some of the techniques described herein, an improved risk prediction model is provided that is configured to perform a model-based risk prediction and treatment pathway prioritization process to overcome technical disadvantages of traditional risk profiling models, thereby improving upon the accuracy, availability, and individual specificity of existing risk prediction techniques while reducing time and processing resources consumed by such techniques.

In some embodiments, a computer-implemented method includes generating, by one or more processors and using a risk prediction model, an individual risk score for an entity of a disparity group associated with an entity cohort; generating, by the one or more processors and using a disparity risk adjustment model, a disparity adjusted risk score for the entity based on the individual risk score; and initiating, by the one or more processors, an initial prediction-based action for the entity based on a comparison between the disparity adjusted risk score and a risk score threshold.

In some embodiments, a computing system includes a memory and one or more processors communicatively coupled to the memory, the one or more processors are configured to generate, using a risk prediction model, an individual risk score for an entity of a disparity group associated with an entity cohort; generate, using a disparity risk adjustment model, a disparity adjusted risk score for the entity based on the individual risk score; and initiate an initial prediction-based action for the entity based on a comparison between the disparity adjusted risk score and a risk score threshold.

In some embodiments, one or more non-transitory computer-readable storage media include instructions that, when executed by one or more processors, cause the one or more processors to generate, using a risk prediction model, an individual risk score for an entity of a disparity group associated with an entity cohort; generate, using a disparity risk adjustment model, a disparity adjusted risk score for the entity based on the individual risk score; and initiate an initial prediction-based action for the entity based on a comparison between the disparity adjusted risk score and a risk score threshold.

### BRIEF DESCRIPTION OF DRAWINGS

FIG. 1 illustrates an example computing system in accordance with one or more embodiments of the present disclosure.

FIG. 2 is a schematic diagram of a management computing entity in accordance with some embodiments discussed herein.

FIG. 3 schematic diagram of a user computing entity in accordance with some embodiments discussed herein.

FIGS. 4A-C provide a dataflow diagram showing example data structures, modules, and operations for performing model-based risk prediction and treatment pathway prioritization in accordance with some embodiments discussed herein.

FIG. 5 is a flowchart showing an example of a process for initiating a prediction-based action based on a disparity adjusted risk score generated for a respective entity in accordance with some embodiments discussed herein.

FIG. 6 is a flowchart showing an example of a process for initiating a subsequent prediction-based action based on an evaluation adjusted risk score generated for a respective entity in accordance with some embodiments discussed herein.

FIG. 7 is a flowchart showing an example of a process for generating a prediction-based action sequence for a respective entity based on a phenotypic profile generated for the respective entity in accordance with some embodiments discussed herein.

### DETAILED DESCRIPTION

Various embodiments of the present disclosure are described more fully hereinafter with reference to the accompanying drawings, in which some, but not all embodiments of the present disclosure are shown. Indeed, the present disclosure may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “example” are used to be examples with no indication of quality level. Terms such as “computing,” “determining,” “generating,” and/or similar words are used herein interchangeably to refer to the creation, modification, or identification of data. Further, “based on,” “based at least in part on,” “based at least on,” “based upon,” and/or similar words are used herein interchangeably in an open-ended manner such that they do not indicate being based only on or based solely on the referenced element or elements unless so indicated. Like numbers refer to like elements throughout. Moreover, while certain embodiments of the present disclosure are described with reference to predictive data analysis, one of ordinary skills in the art will recognize that the disclosed concepts may be used to perform other types of data analysis.

### Computer Program Products, Methods, and Computing Entities

Embodiments of the present disclosure may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query, or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together, such as in a particular directory, folder, or library. Software components may be static (e.g., pre-established, or fixed) or dynamic (e.g., created or modified at the time of execution).

A computer program product may include a non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In some embodiments, a non-volatile computer-readable storage medium may include a floppy disk, flexible disk, hard disk, solid-state storage (SSS) (e.g., a solid-state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAM), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In some embodiments, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It should be appreciated that where embodiments are described to use a computer-readable storage medium, other types of computer-readable storage media may be substituted for, or used in addition to, the computer-readable storage media described above.

As should be appreciated, various embodiments of the present disclosure may also be implemented as methods, apparatuses, systems, computing devices, computing entities, and/or the like. As such, embodiments of the present disclosure may take the form of an apparatus, system, computing device, computing entity, and/or the like executing instructions stored on a computer-readable storage medium to perform certain steps or operations. Thus, embodiments of the present disclosure may also take the form of an entirely hardware embodiment, an entirely computer program product embodiment, and/or an embodiment that comprises combination of computer program products and hardware performing certain steps or operations.

Embodiments of the present disclosure are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some example embodiments, retrieval, loading, and/or execution may be performed in parallel such that multiple instructions are retrieved, loaded, and/or executed together. Thus, such embodiments may produce specifically configured machines performing the steps or operations specified in the block diagrams and flowchart illustrations. Accordingly, the block diagrams and flowchart illustrations support various combinations of embodiments for performing the specified instructions, operations, or steps.

### Example Framework

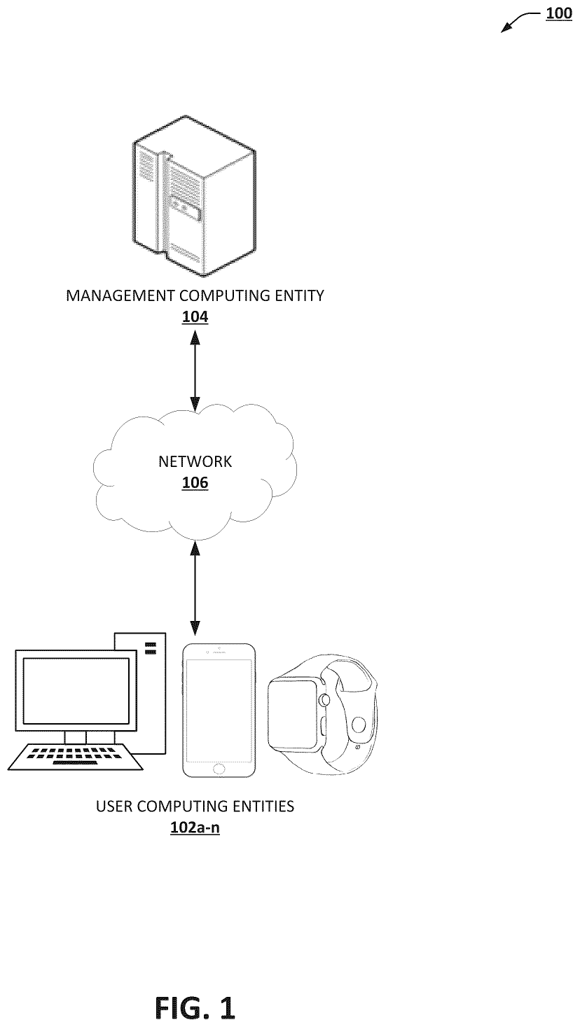

FIG. 1 is a diagram of a computing system 100 that may be used to practice various embodiments of the present disclosure. As shown in FIG. 1, the computing system 100 may include one or more user computing entities 102a-n, one or more management computing entities 104a-n, one or more networks 106, and/or the like. Each of the components of the computing system 100 may be in electronic communication with, for example, one another over the same or different wireless or wired networks 106 including, for example, a wired or wireless Personal Area Network (PAN), Local Area Network (LAN), Metropolitan Area Network (MAN), Wide Area Network (WAN), and/or the like. Additionally, while FIG. 1 illustrates certain system devices as separate, standalone devices, the various embodiments are not limited to this particular architecture.

### Management Computing Entity

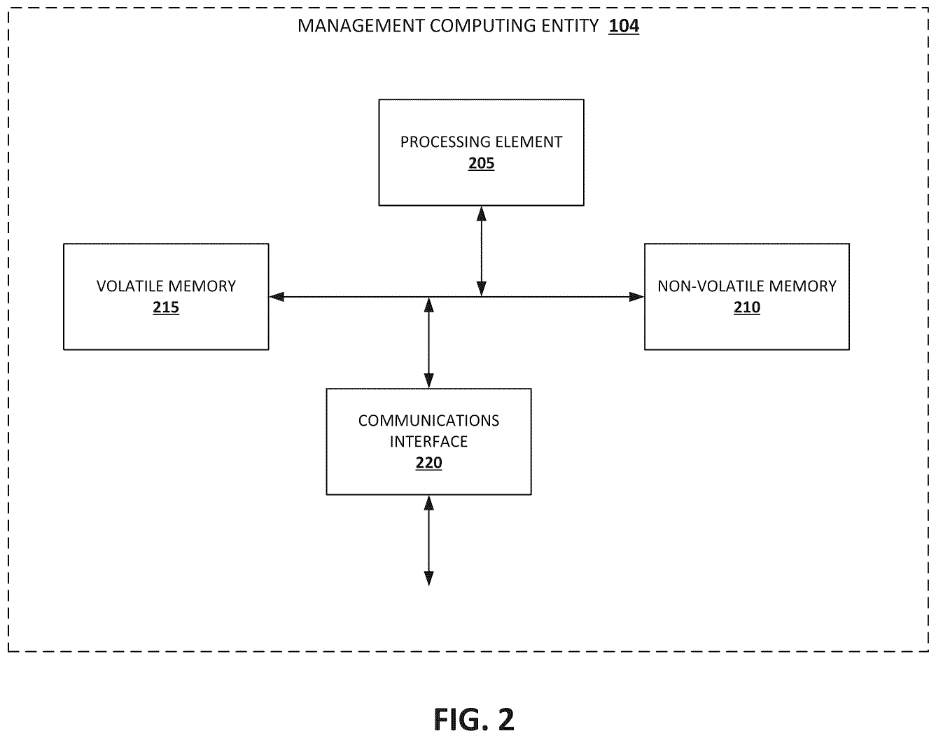

FIG. 2 is a schematic diagram of a management computing entity 104a in accordance with certain embodiments of the present disclosure. In general, the terms computing device, entity, device, system, and/or similar words used herein interchangeably may refer to, for example, one or more computers, computing devices, computing entities, desktop computers, mobile phones, tablets, phablets, notebooks, laptops, distributed systems, terminals, servers or server networks, blades, gateways, switches, processing devices, set-top boxes, relays, routers, network access points, base stations, the like, and/or any combination of devices adapted to perform the functions, operations, and/or processes described herein. Such functions, operations, and/or processes may include, for example, transmitting, receiving, operating on, processing, displaying, storing, determining, generating/creating, monitoring, evaluating, comparing, and/or similar terms used herein interchangeably. In one embodiment, these functions, operations, and/or processes may be performed on data, content, information, and/or similar terms used herein interchangeably.

As indicated, in one embodiment, the management computing entity 104a may also include one or more network and/or communications interfaces 208 for communicating with various computing entities, such as by communicating data, content, information, and/or similar terms used herein interchangeably that may be transmitted, received, operated on, processed, displayed, stored, and/or the like.

As shown in FIG. 2, in one embodiment, the management computing entity 104a may include or be in communication with one or more processing elements 205 (also referred to as processors, processing circuitry, and/or similar terms used herein interchangeably) that communicate with other elements within the management computing entity 104a via a bus, for example. As will be understood, the processing element 202 may be embodied in a number of different ways. For example, the processing element 202 may be embodied as one or more complex programmable logic devices (CPLDs), microprocessors, multi-core processors, coprocessing devices, application-specific instruction-set processors (ASIPs), and/or controllers. Further, the processing element 202 may be embodied as one or more other processing devices or circuitry. The term circuitry may refer to an entirely hardware embodiment or a combination of hardware and computer program products. Thus, the processing element 202 may be embodied as integrated circuits, application specific integrated circuits (ASICs), field programmable gate arrays (FPGAs), programmable logic arrays (PLAs), hardware accelerators, other circuitry, and/or the like. As will therefore be understood, the processing element 202 may be configured for a particular use or configured to execute instructions stored in volatile or non-volatile media or otherwise accessible to the processing element 202. As such, whether configured by hardware or computer program products, or by a combination thereof, the processing element 202 may be capable of performing steps or operations according to embodiments of the present disclosure when configured accordingly.

In one embodiment, the management computing entity 104a may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 204 as described above, such as hard disks, ROM, PROM, EPROM, EEPROM, flash memory, MMCs, SD memory cards, Memory Sticks, CBRAM, PRAM, FeRAM, RRAM, SONOS, racetrack memory, and/or the like. As will be recognized, the non-volatile storage or memory media may store databases, database instances, database management system entities, data, applications, programs, program modules, scripts, source code, object code, byte code, compiled code, interpreted code, machine code, executable instructions, and/or the like. The term database, database instance, database management system entity, and/or similar terms used herein interchangeably may refer to a structured collection of records or information/data that is stored in a computer-readable storage medium, such as via a relational database, hierarchical database, and/or network database.

In one embodiment, the management computing entity 104a may further include or be in communication with volatile media (also referred to as volatile storage, memory, memory storage, memory circuitry and/or similar terms used herein interchangeably). In one embodiment, the volatile storage or memory may also include one or more volatile storage or memory media 206 as described above, such as RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. As will be recognized, the volatile storage or memory media may be used to store at least portions of the databases, database instances, database management system entities, data, applications, programs, program modules, scripts, source code, object code, byte code, compiled code, interpreted code, machine code, executable instructions, and/or the like being executed by, for example, the processing element 202. Thus, the databases, database instances, database management system entities, data, applications, programs, program modules, scripts, source code, object code, byte code, compiled code, interpreted code, machine code, executable instructions, and/or the like may be used to control certain aspects of the operation of the management computing entity 104a with the assistance of the processing element 202 and the operating system.

As indicated, in one embodiment, the management computing entity 104a may also include one or more network and/or communications interfaces 208 for communicating with various computing entities, such as by communicating data, content, information, and/or similar terms used herein interchangeably that may be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, management computing entity 104a may be configured to communicate via wireless external communication networks using any of a variety of protocols, such as general packet radio service (GPRS), Universal Mobile Telecommunications System (UMTS), Code Division Multiple Access 200 (CDMA200), CDMA200 1X (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), IR protocols, NFC protocols, RFID protocols, IR protocols, ZigBee protocols, Z-Wave protocols, 6LoWPAN protocols, Wibree, Bluetooth protocols, wireless universal serial bus (USB) protocols, and/or any other wireless protocol.

The management computing entity 104a may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

As will be appreciated, one or more of the management computing entity's components may be located remotely from other management computing entity 104a components, such as in a distributed system. Furthermore, one or more of the components may be aggregated and additional components performing functions described herein may be included in the management computing entity 104a.

### User Computing Entity

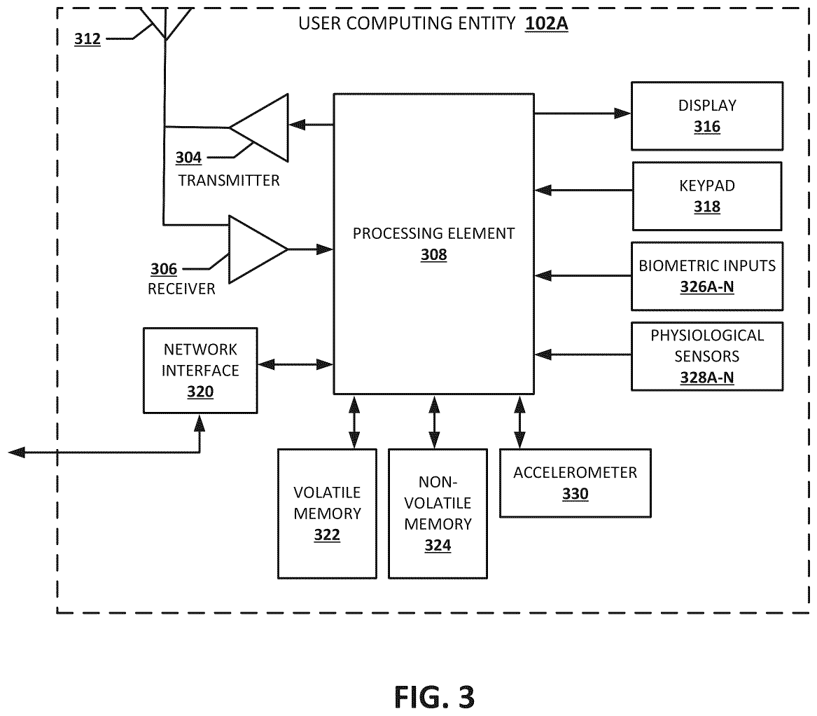

FIG. 3 is a schematic diagram of a user computing entity 102a in accordance with certain embodiments of the present disclosure. In various embodiments, the user computing entity 102a may include one or more computers, computing devices, computing entities, desktop computers, mobile phones, tablets, phablets, notebooks, laptops, mobile devices, wearable computing devices, and/or the like.

As shown in FIG. 3, n user computing entity 102a may include an antenna 312, a transmitter 304 (e.g., radio), a receiver 306 (e.g., radio), and a processing element 308 that provides signals to and receives signals from the transmitter 304 and receiver 306, respectively. The signals provided to and received from the transmitter 304 and the receiver 306, respectively, may include signaling information/data in accordance with an air interface standard of applicable wireless systems to communicate with various devices, such as a management computing entity 104a, another user computing entity 102a, and/or the like. In an example embodiment, the transmitter 304 and/or receiver 306 are configured to communicate via one or more SRC protocols. For example, the transmitter 304 and/or receiver 306 may be configured to transmit and/or receive information/data, transmissions, and/or the like of at least one of Bluetooth protocols, low energy Bluetooth protocols, NFC protocols, RFID protocols, IR protocols, Wi-Fi protocols, ZigBee protocols, ZWave protocols, 6LoWPAN protocols, and/or other short range communication protocol. In various embodiments, the antenna 312, transmitter 304, and receiver 306 may be configured to communicate via one or more long range protocols, such as GPRS, UMTS, CDMA200, 1×RTT, WCDMA, GSM, EDGE, TD-SCDMA, LTE, E-UTRAN, EVDO, HSPA, HSDPA, Wi-Fi, Wi-Fi Direct, WiMAX, and/or the like.

In this regard, the user computing entity 102a may be capable of operating with one or more air interface standards, communication protocols, modulation types, and access types. More particularly, the user computing entity 102a may operate in accordance with any of a number of wireless communication standards and protocols. In a particular embodiment, the user computing entity 102a may operate in accordance with multiple wireless communication standards and protocols, such as GPRS, UMTS, CDMA200, 1×RTT, WCDMA, TD-SCDMA, LTE, E-UTRAN, EVDO, HSPA, HSDPA, Wi-Fi, WiMAX, UWB, IR protocols, Bluetooth protocols, USB protocols, and/or any other wireless protocol.

Via these communication standards and protocols, the user computing entity 102a may communicate with various other devices using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The user computing entity 102a may also download changes, add-ons, and updates, for instance, to its firmware, software (e.g., including executable instructions, applications, program modules), and operating system.

According to one embodiment, the user computing entity 102a may include location determining aspects, devices, modules, functionalities, and/or similar words used herein interchangeably to acquire location information/data regularly, continuously, or in response to certain triggers. For example, the user computing entity 102a may include outdoor positioning aspects, such as a location module adapted to acquire, for example, latitude, longitude, altitude, geocode, course, direction, heading, speed, UTC, date, and/or various other information/data. In one embodiment, the location module may acquire information/data, sometimes known as ephemeris information/data, by identifying the number of satellites in view and the relative positions of those satellites. The satellites may be a variety of different satellites, including LEO satellite systems, DOD satellite systems, the European Union Galileo positioning systems, the Chinese Compass navigation systems, Indian Regional Navigational satellite systems, and/or the like. Alternatively, the location information/data may be determined by triangulating the position of the user computing entity 102a in connection with a variety of other systems, including cellular towers, Wi-Fi access points, and/or the like. Similarly, the user computing entity 102a may include indoor positioning aspects, such as a location module adapted to acquire, for example, latitude, longitude, altitude, geocode, course, direction, heading, speed, time, date, and/or various other information/data. Some of the indoor aspects may use various position or location technologies including RFID tags, indoor beacons or transmitters, Wi-Fi access points, cellular towers, nearby computing entities (e.g., smartphones, laptops) and/or the like. For instance, such technologies may include iBeacons, Gimbal proximity beacons, BLE transmitters, NFC transmitters, and/or the like. These indoor positioning aspects may be used in a variety of settings to determine the location of someone or something to within inches or centimeters.

The user computing entity 102a may also include a user interface device comprising one or more user input/output interfaces (e.g., a display 316 and/or speaker/speaker driver coupled to a processing element 308 and a touch interface, keyboard, mouse, and/or microphone coupled to a processing element 308). For example, the user interface may be configured to provide an application (e.g., mobile app), browser, interactive user interface, dashboard, webpage, and/or similar words used herein interchangeably executing on and/or accessible via the user computing entity 102a to cause display or audible presentation of information/data and for user interaction therewith via one or more user input interfaces. In one embodiment, the functionality described herein (and user interface) may be provided as a standalone app executing on the user computing entity 102a. In such an implementation, the standalone app may be integrated with a variety of other apps executing on the user computing entity 102a to provide authentication functionality for other apps. Moreover, the user interface may include or be in communication with any of a number of devices allowing the user computing entity 102a to receive information/data, such as a keypad 318 (hard or soft), a touch display, voice/speech or motion interfaces, scanners, readers, or other input device. In embodiments including a keypad 318, the keypad 318 may include (or cause display of) the conventional numeric (0-9) and related keys (#, *), and other keys used for operating the user computing entity 102a and may include a full set of alphabetic keys or set of keys that may be activated to provide a full set of alphanumeric keys. In addition to providing input, the user input interface may be used, for example, to activate or deactivate certain functions, such as screen savers and/or sleep modes. Through such inputs, the user computing entity 102a may capture, collect, store information/data, user interaction/input, and/or the like.

In various example embodiments, the user computing entity 102a may include one or more biometric input components 326a-n (e.g., sensors, elements) for receiving or capturing biometric inputs or information/data (e.g., regularly, continuously, or in response to certain triggers). For example, the user computing entity 102a may include a touch sensitive region and/or display for capturing fingerprint scans, in an example embodiment. In another example, the user computing entity 102a may include cameras and/or image capturing devices for capturing images (e.g., image information/data) of an iris and/or face to determine blink rates or skin responses and/or detect coughing episodes. In another example, the user computing entity 102a may include microphones for capturing cough samples for cough detection and recognition. As should be understood, the user computing entity 102a may include various biometric input components 326a-n (e.g., sensors, elements) for receiving biometric input and information/data from a user. In various example embodiments, the user computing entity 102a may regularly, continuously, or in response to certain triggers capture such information/data (e.g., image information/data and/or biometric information/data).

In another example embodiment, the user computing entity 102a may include one or more physiological components 328a-n (e.g., sensors, elements) for capturing physiological inputs or information/data (e.g., regularly, continuously, or in response to certain triggers). For example, the user computing entity 102a may include microelectromechanical (MEMS) components, biological and chemical sensing components, electrocardiogram (ECG) components, electromyogram (EMG) components, electroencephalogram (EEG)-based neural sensing components, optical sensing components, electrical sensing components, sound components, vibration sensing components, and/or the like. Through such components, various types of physiological information/data may be captured-such as heart rate information/data, oxygen saturation information/data, carbon dioxide information/data, temperature information/data, breath rate information/data, perspiration information/data, neural information/data, cardiovascular sounds information/data, pulmonary sounds information/data, and/or various other types of information/data.

In another example embodiment, the user computing entity 102a may include one or more accelerometers, gyroscopes, and/or inertial measurement units (referred to herein separately and collectively as accelerometers 330) for capturing accelerometer information/data. For example, the accelerometers may capture static and dynamic acceleration, angular velocity, and degrees of freedom (DOF) to provide highly accurate orientation, position, and velocity information/data (e.g., accelerometer information/data).

The user computing entity 102a may also include volatile storage or memory 322 and/or non-volatile storage or memory 324, which may be embedded and/or may be removable. For example, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory may store databases, database instances, database management system entities, information/data, applications, programs, program modules, scripts, source code, object code, byte code, compiled code, interpreted code, machine code, executable instructions, and/or the like to implement the functions of the user computing entity 102a.

### Networks

In one embodiment, any two or more of the illustrative components of the computing system 100 of FIG. 1 may be configured to communicate with one another via one or more networks 106. The networks 106 may include, but are not limited to, any one or a combination of different types of suitable communications networks such as cable networks, public networks (e.g., the Internet), private networks (e.g., frame-relay networks), wireless networks, cellular networks, telephone networks (e.g., a public switched telephone network), or any other suitable private and/or public networks. Further, the networks 106 may have any suitable communication range associated therewith and may include, for example, global networks (e.g., the Internet), MANs, WANs, LANs, or PANs. In addition, the networks 106 may include any type of medium over which network traffic may be carried including coaxial cable, twisted-pair wire, optical fiber, a hybrid fiber coaxial (HFC) medium, microwave terrestrial transceivers, radio frequency communication mediums, satellite communication mediums, or any combination thereof, as well as a variety of network devices and computing systems provided by network providers or other entities.

### Examples of Certain Terms

In some embodiments, the term “risk prediction model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A risk prediction model may include one or more machine learning models configured, trained (e.g., jointly, separately, etc.), and/or the like to generate individual risk scores, disparity adjusted risk scores, evaluation adjusted risk scores, recommendations, and/or phenotypic profiles. The risk prediction model may also be configured to initiate and/or cause execution of one or more prediction-based actions for one or more respective entities associated with a respective disparity group, entity cohort, and/or historical entity datastore (e.g., an electronic medical record (EMR) datastore). A risk prediction model may include one or more of any type of machine learning model including one or more supervised, unsupervised, semi-supervised, reinforcement learning models, and/or the like. In some examples, a risk prediction model may include multiple models configured to perform one or more different stages of a prediction process.

For example, in some embodiments, a risk prediction model may comprise, embody, integrate with, employ, and/or otherwise be associated with one or more of a disparity risk adjustment model, an evaluation risk adjustment model, and/or a multi-dimensional location ranking model. Additionally or alternatively, a risk prediction model may be configured to apply predictive modelling techniques such as logistic regression, Reverse Time AttnetIoN (RETAIN), and/or gradient boosting. In some embodiments, a risk prediction model is trained using EMR data related to a one or more entities, sets of patients, and/or disparity groups.

In some embodiments, a risk prediction model may, based on an individual risk score, disparity adjusted risk score, or evaluation adjusted risk score associated with a respective entity (e.g., a respective medical patient), generate, receive, and/or manage one or more data objects related to the initiation and/or execution of one or more prediction-based actions, instructions, and/or commands related to the respective entity. For example, a disparity adjusted risk score may be evaluated in comparison to one or more risk score thresholds to determine whether the respective entity is to be categorized as a high, medium, or low risk for a respective disease, condition, malady, sickness, disorder, and/or impairment. Based on the one or more risk score thresholds, the risk prediction model may generate, receive, and/or manage one or more data objects related to the initiation and/or execution of one or more prediction-based actions, instructions, and/or commands related to the respective entity.

For example, if a disparity adjusted risk score indicates that a respective entity is at a low risk for a particular disease, condition, malady, sickness, disorder, and/or impairment, the risk prediction model may initiate an entity evaluation cessation action associated with the respective entity. In some embodiments, the entity evaluation cessation action is associated with a predetermined periodicity of time for which the entity is not further computationally evaluated with respect to the particular disease, condition, malady, sickness, disorder, and/or impairment related to the disparity adjusted risk score.

As another example, if a disparity adjusted risk score indicates that a respective entity is at a medium risk for a particular disease, condition, malady, sickness, disorder, and/or impairment, the risk prediction model may initiate an entity monitoring action associated with the respective entity. In some embodiments, the entity monitoring action is an employment of an entity monitoring computing device to collect entity monitoring data associated with the respective entity (e.g., data related to cognitive health, heart health, renal health, diabetic health, and/or the like). As such, in some embodiments, based on the entity monitoring action, the risk prediction model may cause association (e.g., issuance, delivery, network linkage, and/or monitoring) of an entity monitoring computing device with the respective entity.

As another example, if a disparity adjusted risk score indicates that a respective entity is at a high risk for a particular disease, condition, malady, sickness, disorder, and/or impairment, the risk prediction model may initiate an entity evaluation action associated with the respective entity. In some embodiments, the entity evaluation action is associated with one or more physical, mental, and/or emotional evaluations related to the respective disease, condition, malady, sickness, disorder, and/or impairment associated with the disparity adjusted risk score. For example, in some embodiments, the entity evaluation action is associated with a cognitive evaluation for the entity, where the cognitive evaluation is at least one of a computer-scored and/or specialist-scored text-based entity evaluation (e.g., a handwriting evaluation, reading comprehension evaluation, memory evaluation, written evaluation, and/or the like), a speech-based entity evaluation (e.g., an evaluation to detect whether an entity is slurring, mispronouncing, and/or stuttering while speaking, and/or an interview-style evaluation administered to an entity), a gait-based entity evaluation (e.g., a walking evaluation, balance evaluation, standing evaluation, sitting evaluation, and/or the like), or a biomarker-based evaluation (e.g., a blood pressure evaluation, vascular evaluation, glucose evaluation, cerebral-spinal fluid evaluation, and/or the like). Furthermore, in some embodiments, the risk prediction model may generate correspondence associated with the entity evaluation action (e.g., informational correspondence, scheduling correspondence, insurance correspondence, etc.) and cause transmission, delivery, notification, and/or data indicative of (e.g., including one or more evaluation identifiers, etc.) the correspondence associated with the entity evaluation action to the respective entity.

In some embodiments, the term “individual risk score” refers to a data value that describes an individual entity's likelihood of having or developing a particular disease, condition, malady, sickness, disorder, and/or impairment (e.g., dementia, cognitive impairment, heart disease, diabetes, and/or the like). For example, an individual risk score may include a real number, probability value, percentage, and/or the like that describes an entity-level (e.g., member-level for a clinical domain) probability of being associated with a particular disease. For example, an individual risk score may include a value within a defined range, such as zero to one, in which a first value (e.g., 0) of the defined range indicates a low likelihood of being associated with a particular disease and a high value (e.g., 1) indicates a high likelihood of being associated with the particular disease. In some examples, an individual risk score may include a high value (e.g., 1 in a defined range of 0 to 1) in the event that an entity is associated with a currently documented particular disease. In some examples, an individual risk score may include a value within the defined range in the event that an entity is not associated with a currently documented disease.

In some embodiments, an individual risk score is generated by a risk prediction model based on historical records (e.g., medical records) related to a respective entity stored in a historical entity datastore. For example, an individual risk score may be configured to measure a likelihood of an individual entity having or developing a particular disease based on an EMR dataset associated with the individual entity stored in a historical entity datastore, where the EMR dataset comprises at least one or more of a personal medical history, a family medical history, a disease risk factor, a head trauma history and/or the like previously recorded for an individual entity. In some examples, an individual risk score may be generated for each entity of a disparity group. Furthermore, in some embodiments, an individual risk score may be updated and/or adjusted by a disparity risk adjustment model associated with the risk prediction model to generate a disparity adjusted risk score for a respective entity.

In some embodiments, the term “disparity risk adjustment model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A disparity risk adjustment model may include one or more of any type of machine learning model including one or more supervised, unsupervised, semi-supervised, reinforcement learning models, and/or the like. In some embodiments, a disparity risk adjustment model may include one or more machine learning models configured, trained (e.g., jointly, separately, etc.), and/or the like to generate a disparity adjusted risk score associated with a respective entity (e.g., a respective medical patient).

For example, in some embodiments, the disparity adjusted risk score associated with the respective entity is generated based on performing a disparity risk adjustment. The disparity risk adjustment may comprise generating, using the risk prediction model, a risk prevalence ratio, where the risk prevalence ratio is a ratio of a documented risk prevalence associated with a disparity group to an estimated risk prevalence associated with the disparity group. In some embodiments, the estimated risk prevalence is determined based on aggregating the individual risk scores for one or more respective entities in the disparity group associated with a respective entity cohort. As such, the disparity adjusted risk score may be generated based on applying at least the risk prevalence ratio and an entity-defined disparity weighting parameter to the individual risk score associated with the entity. Additionally or alternatively, in some embodiments, an individual risk score associated with the respective entity may be updated based on entity monitoring data generated by an entity monitoring computing device associated with the individual entity.

In some embodiments, performing the disparity risk adjustment may comprise executing a disparity risk adjustment equation defined as:

$$P ( D ) = {e}^{log ( P ( D - 1 ) ) + ( β \times log ( 1 - R ) )} \qquad Equation 1$$

where P(D) is the disparity adjusted risk score for the respective entity, P(D−1) is the initial individual risk score for the respective entity, β is a user-defined disparity weighting parameter (e.g., having possible values are between 0 and 1), and R is a risk prevalence ratio, where the risk prevalence ratio is a ratio of a documented risk prevalence associated with the disparity group to an estimated risk prevalence associated with the disparity group. Generating disparity adjusted risk scores for respective entities has the effect of prioritizing entities in disparity groups where there is a greater level of undiagnosed adverse risks, situations, outcomes, and/or conditions (e.g., diseases, conditions, maladies, sicknesses, disorders, and/or impairments).

In some embodiments, the term “disparity adjusted risk score” to refers to a data entity generated by a risk prediction model for an entity associated with an entity cohort and/or a disparity group and is generated based on a disparity risk adjustment. The disparity adjusted risk score may indicate if an entity is at a high, medium, or low risk of developing, experiencing, and/or undergoing an adverse outcome, situation, and/or condition associated with one or more technical domains (e.g., healthcare domains, financial domains, insurance domains, and/or the like). For example, in the context of the healthcare domain, a disparity adjusted risk score may indicate if an entity is at a high, medium, or low risk of having and/or developing, a particular disease, disorder, and/or impairment, such as cognitive impairment, dementia, heart disease, diabetes, renal disease, and/or the like.

A disparity adjusted risk score may include an imputed probability score (e.g., an imputed disease probability score for a clinical domain, etc.). A disparity adjusted risk score, for example, may be configured to measure a likelihood of a particular disease based on one or more entity attributes of an individual entity (e.g., as recorded by an entity data object, etc.). In some examples, a disparity adjusted risk score may be generated for each entity of a disparity group associated with a respective entity cohort.

In some embodiments, a disparity adjusted risk score is generated, using one or more machine learning-based techniques, based on entity attributes associated with an entity. For example, a disparity adjusted risk score may include a projection of a new and unique risk that may emerge for an individual entity. In some examples, the disparity adjusted risk score may be generated using time series prediction techniques that generate a risk score based on entity attributes (e.g., clinical and/or non-clinical information about the members in a clinical domain) to predict emerging risk for a particular disease.

In some embodiments, a disparity adjusted risk score may be used by a risk prediction model to generate one or more recommendations and/or cause execution of one or more actions, instructions, and/or commands related to a respective entity (e.g., a respective patient). For example, a disparity adjusted risk score may be evaluated (e.g., via the risk prediction model) in comparison to one or more risk score thresholds to determine whether the respective entity is to be categorized as a high, medium, or low risk for a particular disease, condition, malady, sickness, disorder, and/or impairment. Based on the one or more risk score thresholds, the risk prediction model may generate, receive, and/or manage one or more data objects related to the initiation and/or execution of one or more prediction-based actions, instructions, and/or commands related to the respective entity. Furthermore, in some embodiments, a disparity adjusted risk score may be updated and/or adjusted by an evaluation risk adjustment model based on an evaluation data object associated with the respective entity generated based on an entity evaluation action.

In some embodiments, the term “disparity group” to refers to a data entity that describes a subset of entities within an entity cohort. An entity may be any person that is associated with a particular entity cohort. A disparity group may include a subset of a plurality of entities within the particular entity cohort that are grouped based on one or more criteria. By way of example, a disparity group may include a subset of entities that share one or more common and/or related attributes that are defined by one or more criteria. For example, a disparity group may be associated with a particular geographic region. Additionally, a disparity group may be associated with one or more contextual attributes. The one or more contextual attributes may be associated with one or more demographic characteristics (e.g., race, ethnicity, gender, age, lifestyle choices, and/or the like), membership plans (e.g., healthcare plan, insurance plan, etc.), and/or may describe one or more common entity experiences (e.g., access to healthcare, access to doctors, access to specialists, access to transportation, financial needs, and/or the like).

In some embodiments, the term “entity data object” refers to a data entity that describes an entity within a disparity group, entity cohort, historical entity datastore (e.g., an EMR datastore), and/or the like. An entity data object, for example, may include a plurality of entity attributes for an entity.

In some embodiments, the term “contextual attribute” refers to a data parameter that describes a characteristic of an entity associated with a respective entity data object. A contextual attribute, for example, may include a current attribute that is descriptive of one or more current characteristics of an entity, such as one or more demographic characteristics (e.g., race, ethnicity, gender, age, lifestyle choices, and/or the like), location characteristics, membership plans (e.g., healthcare plan, insurance plan, etc.), and/or may describe one or more entity experiences (e.g., access to healthcare, access to doctors, access to specialists, access to transportation, financial needs, and/or the like). In addition, or alternatively, a contextual attribute may include a historical attribute that is descriptive of one or more historical characteristics of an entity, such as one or more recorded events, and/or the like. An entity attribute may depend on a prediction domain. As one example, in a clinical domain, entity attributes may include one or more current attributes, such as current (e.g., within a current year) medical diagnoses, residential location, one or more demographic classes, and/or the like. In addition, or alternatively, in a clinical domain, contextual attributes may include one or more historical parameters and/or attributes, such as historical (e.g., preceding a current year) medical diagnoses, clinical encounters (e.g., hospital visits, etc.), and/or the like.

In some embodiments, the term “historical parameter” refers to a data entity that describes a historical attribute for an entity. A historical parameter, for example, may include a parameter that is previously assigned to an entity as reflected by an entity data object. A historical parameter may include any type of parameter depending on the prediction domain, including clinical parameters (e.g., medications, diagnoses, etc.) for a clinical domain, financial parameters (e.g., financial transactions, lending activities, etc.) for a financial domain, and/or the like. As an example, in a clinical domain, a historical parameter may include a medication prescription and/or a diagnosis for a patient. For example, an entity data object may include a patient record with a plurality of existing and/or previously prescribed (e.g., within a threshold time range, etc.) medications, conditions, and/or the like for a patient. In some embodiments, one or more historical parameters are considered to determine one or more individual risk scores, disparity adjusted risk scores, and/or evaluation adjusted risk scores for an entity.

In some embodiments, the term “prediction-based action” refers to a computing action initiated, triggered, executed, and/or caused by a risk prediction model. By way of example, a prediction-based action may include an entity evaluation action. For example, in some embodiments, the entity evaluation action is associated with a cognitive evaluation for the entity, where the cognitive evaluation is associated with at least one of a computer-scored and/or specialist-scored text-based entity evaluation (e.g., a handwriting evaluation, reading comprehension evaluation, memory evaluation, written evaluation, and/or the like), a speech-based entity evaluation (e.g., an evaluation to detect whether an entity is slurring, mispronouncing, and/or stuttering while speaking, and/or an interview-style evaluation administered to an entity), a gait-based entity evaluation (e.g., a walking evaluation, balance evaluation, standing evaluation, sitting evaluation, and/or the like), or a biomarker-based evaluation (e.g., a blood pressure evaluation, vascular evaluation, glucose evaluation, cerebral-spinal fluid evaluation, and/or the like). As another example, a prediction-based action may include an image-based evaluation action. For example, in some embodiments, an image-based evaluation action may be an imaging action such as a magnetic resonance imaging (MRI) procedure, a fluid attenuated inversion recovery (FLAIR) sequence, positron emission tomography (PET) procedure, or computed tomography (CT) procedure performed with respect to one or more body parts of an entity. As another example, a prediction-based action may include an initiation of an alert, notification, referral, and/or other correspondence to be transmitted, delivered, and/or otherwise conveyed to an entity, medical professional, healthcare administrator, membership plan administrator, and/or the like.

In some embodiments, the term “evaluation risk adjustment model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A disparity risk adjustment model may include one or more of any type of machine learning model including one or more supervised, unsupervised, semi-supervised, reinforcement learning models, and/or the like. In some embodiments, an evaluation risk adjustment model may include one or more machine learning models configured, trained (e.g., jointly, separately, etc.), and/or the like to generate an evaluation adjusted risk score associated with a respective entity (e.g., a respective medical patient).

For example, in some embodiments, the evaluation adjusted risk score associated with the respective entity is generated based on performing an evaluation risk adjustment. In some embodiments, the evaluation risk adjustment is performed by the evaluation adjustment risk model in response to determining that a disparity adjusted risk score associated with a respective entity satisfies a high risk score threshold indicating that the respective entity is at a high risk for a particular disease, condition, malady, sickness, disorder, and/or impairment.

The evaluation adjustment risk model may generate the evaluation adjusted risk score for the entity based on the disparity adjusted risk score and an evaluation data object associated with the entity, where the evaluation data object is generated based on the entity evaluation action, and where the evaluation data object may include data related to one or more evaluations of the entity (e.g., cognitive evaluation scores, results, and/or other data). In some embodiments, the evaluation data comprised in an evaluation data object associated with a respective entity may be compared to normative median evaluation values (e.g., median evaluation values associated with entities that do not, or will not, have the particular disease, condition, malady, sickness, disorder, and/or impairment the respective entity is at risk for).

In some embodiments, the evaluation data object may include cognitive evaluation scores associated with the respective entity, and the cognitive evaluation scores may be divided by a normative median cognitive evaluation score. As such, the resulting cognitive evaluation scores may be employed in the evaluation risk adjustment in order to prioritize entities associated with a disparity group and/or entity cohort with lower cognitive evaluation scores for subsequent prediction-based actions (e.g., an image-based entity evaluation action). For example, the evaluation adjusted risk score for a respective entity may be compared to one or more risk score thresholds to determine whether to generate one or more subsequent prediction-based actions associated with the entity.

In some embodiments, the term “evaluation adjusted risk score” to refers to a data entity generated by a risk prediction model for an entity associated with an entity cohort and/or a disparity group and is generated based on an evaluation risk adjustment. The evaluation adjusted risk score may indicate if a patient is at a high, medium, or low risk of having or developing a particular disease, condition, malady, sickness, disorder, and/or impairment (e.g., dementia, cognitive impairment, heart disease, diabetes, and/or the like). For example, an evaluation adjusted risk score may include an imputed probability score (e.g., an imputed disease probability score for a clinical domain, etc.). An evaluation adjusted risk score, for example, may be configured to measure a likelihood of a particular disease based on one or more entity attributes of an individual entity (e.g., as recorded by an entity data object, etc.). In some examples, an evaluation adjusted risk score may be generated for each entity of a disparity group associated with a respective entity cohort.

In some embodiments, an evaluation adjusted risk score is generated, using one or more machine learning-based techniques, based on entity attributes associated with an entity. For example, an evaluation adjusted risk score may include a projection of a new and unique risk that may emerge for an individual entity. In some examples, the evaluation adjusted risk score may be generated using time series prediction techniques that generate a risk score based on entity attributes (e.g., clinical and/or non-clinical information about the members in a clinical domain) to predict emerging risk for a particular disease.

In some embodiments, an evaluation adjusted risk score may be used by a risk prediction model to generate, receive, and/or manage one or more data objects related to the initiation and/or execution of one or more prediction-based actions, instructions, and/or commands related to the respective entity (e.g., a respective patient). For example, an evaluation adjusted risk score may be evaluated (e.g., via the risk prediction model) in comparison to one or more risk score thresholds to determine whether the respective entity is to be categorized as a high, medium, or low risk for a particular disease, condition, malady, sickness, disorder, and/or impairment. Based on the one or more risk score thresholds, the risk prediction model may generate, receive, and/or manage one or more data objects related to the initiation and/or execution of one or more prediction-based actions, instructions, and/or commands related to the respective entity.

In some embodiments, the term “entity rank” to refers to a data entity generated by a multi-dimensional location ranking model for an entity associated with an entity cohort and/or a disparity group. In some embodiments, an entity rank is generated based on data associated with one or more of an evaluation data object generated based on an entity evaluation action (e.g., a cognitive evaluation) and/or an image-based evaluation data object generated based on an image-based evaluation action (e.g., an MRI evaluation) associated with a respective entity. The multi-dimensional location ranking model may be configured to map an entity rank associated with the respective entity in a multi-dimensional disease space in order to generate a phenotypic profile for the respective entity.

In some embodiments, the term “multi-dimensional location ranking model” refers to a data entity that describes parameters, hyper-parameters, and/or defined operations of a rules-based and/or machine learning model (e.g., model including at least one of one or more rule-based layers, one or more layers that depend on trained parameters, coefficients, and/or the like). A multi-dimensional location ranking model may include one or more of any type of machine learning model including one or more supervised, unsupervised, semi-supervised, reinforcement learning models, and/or the like. In some embodiments, a multi-dimensional location ranking model may include one or more machine learning models configured, trained (e.g., jointly, separately, etc.), and/or the like to generate an entity rank for an entity associated with an entity cohort and/or a disparity group. Furthermore, the multi-dimensional location ranking model may be configured to perform multi-dimensional location ranking via Euclidean distance in order to facilitate the mapping of an entity rank associated with the respective entity in a multi-dimensional disease space. By mapping the entity rank in the multi-dimensional disease space, the multi-dimensional location ranking model may generate a phenotypic profile for the respective entity.

In some embodiments, median normative evaluation data and median image-based evaluation data provide the basis for multi-dimensional location ranking via Euclidean distance. For example, the evaluation data comprised in an evaluation data object and/or image data comprised in an image-based evaluation data object associated with a respective entity may be compared to normative median evaluation values (e.g., median evaluation values associated with entities that do not, or will not, have the particular disease, condition, malady, sickness, disorder, and/or impairment the respective entity is at risk for).

In some examples, while the evaluation data comprised in an evaluation data object may already be in a numerical form, the image data comprised in an image-based evaluation data object (e.g., brain MRI results rendered in a 3-dimensional image format) may require additional preprocessing before the image data may be employed in multi-dimensional location ranking. In examples related to the cognitive health domain, brain tissue and lobar segmentation may be performed to transform brain image data related to a respective entity into a numerical form. In such examples, the proportion of brain tissue (e.g., gray matter and white matter) per brain lobe (e.g., frontal, parietal, temporal, and/or occipital lobes) may be calculated on an individual entity basis and groupwise median values may be obtained from a normative population. In some embodiments, if PET is performed on a respective entity, average and/or median PET centiloid values may also be computed per lobe.

In some embodiments, the multi-dimensional location ranking model may be configured to perform multi-dimensional location ranking via Euclidean distance for a plurality of domain data (e.g., imaging, cognition, blood, and/or other biomarker data that is clinically indicated) via a multi-dimensional location ranking equation, defined as:

$$r = \sqrt{{( {i}_{n} - {i}_{p} )}^{2} + … + {( {C}_{n} - {C}_{p} )}^{2}} \qquad Equation 2$$

where r is an entity rank associated with a distance from a multi-dimensional normative median, iₙ is a normative median imaging value, iₚ is an imaging value associated with a particular entity (e.g., imaging data associated with an image-based evaluation data object), Cₙ is a normative median cognition value, and Cₚ is an entity cognition evaluation value (e.g., cognitive evaluation value). The coefficients i and C may be single numbers or vectors corresponding to multiple evaluations within each respective domain. In some embodiments, additional domains (e.g., indicated by “ . . . ”) may be added to the multi-dimensional location ranking equation in the same form as presented above for i and C, (e.g., an addition of the square of entity values subtracted from normative median values).

The value for r may provide a degree of disease severity and/or location in a multi-dimensional disease space. Each of the individual domains associated with the coefficients considered in the multi-dimensional location ranking equation (e.g., i and C) may be associated with the multi-dimensional disease space such that an assessment of a distance value (e.g., Euclidian distance value) of each individual domain may be employed by the multi-dimensional location ranking model to determine a phenotypic profile for a respective entity. For example, in the cognitive health domain, the values for r, i, and C may suggest a particular dementia subtype (e.g., a large value for r coupled with long distance values for cognitive behavioral problems and frontal and/or temporal brain tissue loss, e.g., as evidenced by image data, may indicate a behavioral variant frontotemporal dementia phenotypic profile for a respective entity).

In some embodiments, the term “phenotypic profile” refers to a data entity associated with a respective entity related to a respective disparity group and/or a respective entity cohort. In some embodiments, a phenotypic profile comprises at least one of a disease severity and/or a disease subtype associated with the respective entity. The disease severity and/or the disease subtype may characterize, describe, and/or indicate one or more attributes related to a respective disease, condition, malady, sickness, disorder, and/or impairment associated with the respective entity. Furthermore, the phenotypic profile may describe whether a current condition of the respective entity is a normal condition, reversible condition, or irreversible condition.

In some embodiments, the term “prediction-based action sequence” refers to a data entity associated with a respective entity related to a respective disparity group and/or a respective entity cohort. In some embodiments, the prediction-based action sequence may be associated with one or more prediction-based actions to be initiated, executed, and/or performed with respect to the respective entity based on a phenotypic profile associated with the respective entity. For example, the risk prediction model may generate a particular prediction-based action sequence for the respective entity based on a determination related to whether the phenotypic profile of the entity describes that a current condition of the respective entity is a normal condition, a reversible condition, or an irreversible condition. In some embodiments, the prediction-based action sequence may be associated with a particular treatment plan and/or set of recommendations configured to treat, alleviate, address, mitigate, and/or otherwise manage a particular disease, condition, malady, sickness, disorder, and/or impairment associated with the respective entity.

For example, if a phenotypic profile of a respective entity indicates that a current condition related to the entity is a normal condition, the risk prediction model may generate a prediction-based action sequence comprising an entity evaluation cessation action associated with the respective entity. In some embodiments, the entity evaluation cessation action is associated with a predetermined periodicity of time for which the entity is not further computationally evaluated with respect to the particular disease, condition, malady, sickness, disorder, and/or impairment related to the respective entity.

As another example, if a phenotypic profile of a respective entity indicates that a current condition related to the entity is a reversible condition, the risk prediction model may generate a prediction-based action sequence comprising a condition investigation action associated with the particular disease, condition, malady, sickness, disorder, and/or impairment associated with the respective entity. For example, in the cognitive health domain, entities with potentially reversible cognitive impairment (e.g., entities without atrophy, small vessel disease, and/or other biomarker involvement) may be referred for additional evaluations that are tangentially related to a current condition of the entity (e.g., evaluations associated with mental disorders (e.g., clinical depression), delirium, hydrocephalus and/or the like).

As another example, if a phenotypic profile of a respective entity indicates that a current condition related to the entity is an irreversible condition, the risk prediction model may generate a prediction-based action sequence comprising a specialist referral monitoring action associated with the respective entity. In such examples, the risk prediction model may generate a specialist referral associated with a respective medical specialist based on the specialist referral action. Furthermore, in some embodiments, the risk prediction model may generate correspondence associated with the specialist referral (e.g., informational correspondence, scheduling correspondence, insurance correspondence, etc.) and cause transmission, delivery, notification, and/or data indicative of (e.g., including one or more referral identifiers, etc.) the correspondence associated with the specialist referral to the respective entity. For example, in the cognitive health domain, entities with irreversible cognitive impairment (e.g., entities with severe brain tissue atrophy and/or a large amyloid plaque burden) may be referred to a relevant neurologist, old-age psychiatrist, and/or care facility.

### Overview

Embodiments of the present disclosure address technical challenges related to computer-based risk predictions for individual entities that have developed, or are at risk of developing, experiencing, and/or undergoing an adverse outcome, situation, and/or condition associated with one or more technical domains (e.g., healthcare domains, financial domains, insurance domains, and/or the like). For example, in the context of the healthcare domain, embodiments of the present disclosure address technical challenges related to computer-based risk predictions for individual entities that have developed, or are at risk of developing, a particular disease, disorder, and/or impairment, such as cognitive impairment, dementia, heart disease, diabetes, renal disease, and/or the like.

According to the World Health Organization, over 50 million people are living with dementia and this number is expected to double every twenty years. However, the majority of these cases are undiagnosed. This is partly due to primary care being the gateway to secondary investigations by medical specialists that may be required for final diagnosis. Limited primary care capacity has led to bottlenecks in service provision, variability in care pathways, and delayed diagnoses. Additionally, existing computer-based techniques for determining an individual risk that a respective entity (e.g., a medical patient) will develop a particular disease, ailment, and/or impairment are lacking in that the existing techniques do not generate a treatment pathway for an entity that is at high risk.

Embodiments of the present disclosure present proactive and automatic model-based risk prediction techniques to assess the individual risk associated with the individual entities associated with a respective disparity group and/or entity cohort (e.g., a cohort of medical patients, etc.) and initiate prediction-based actions related to the individual entities. To do so, a risk prediction model is trained and employed to integrate total risk prevalence (e.g., disease risk prevalence, etc.) and/or prevalence disparity statistics to address regional and/or demographic inequalities related to various domains (e.g., healthcare domains, financial domains, and/or the like). In this regard, embodiments of the present disclosure are configured to employ various ML models configured to generate and adjust individual risk scores associated with respective entities of a disparity group in order to generate individual, prediction-based actions and/or prediction-based action sequences for the respective entities.

Embodiments of the present disclosure provide a multi-modal approach to predicting and/or mitigating the individual risk of the respective entities. For example, embodiments of the present disclosure provide a risk prediction model configured to embody, integrate with, manage, and/or otherwise employ one or more of a disparity risk adjustment model, evaluation risk adjustment model, and/or a multi-dimensional location ranking model. The various ML models associated with the risk prediction model are configured to generate individual risk scores, disparity adjusted risk scores, evaluation adjusted risk scores, prediction-based actions, prediction-based recommendations, and/or phenotypic profiles for one or more entities associated with the respective disparity group, entity cohort, and/or historical entity datastore. The risk prediction model may also be configured to initiate and/or cause execution of the one or more prediction-based actions for one or more respective entities based on a comparison of one or more of the disparity adjusted risk score and/or the evaluation adjusted risk score associated with a respective entity to one or more risk score thresholds. In this regard, the risk prediction model may initiate and/or facilitate the execution of one or more entity evaluation actions, image-based entity evaluation actions, entity monitoring actions, and/or entity evaluation cessation actions.

Embodiments of the present disclosure are configured to facilitate the performance of staged evaluations and reintegrate the resulting evaluation data back into individual risk data prior to suggesting, generating, and/or causing performance of diagnostic and/or mitigative decisions. As such, evaluation data (e.g., clinical evaluation scores, etc.) associated with a respective entity may be stored in a historical entity datastore and used as predictive modelling features. Additionally, embodiments of the present disclosure provide techniques for the automated integration of remote monitoring data (e.g., health data collected via a computing device configured to monitor a respective entity) into individual risk scores.

Embodiments of the present disclosure are also configured to generate, via the risk prediction model, a phenotypic profile for a respective entity. A phenotypic profile may describe a risk subtype and/or risk severity (e.g., a disease risk subtype and/or severity) associated with the respective entity. Furthermore, the risk prediction model may be configured to generate individual, prediction-based action sequences based on the phenotypic profile associated with the respective entity. In a healthcare context, the prediction-based action sequences may be associated with a particular treatment plan and/or set of recommendations configured to treat, alleviate, address, mitigate, and/or otherwise manage a particular disease, condition, malady, sickness, disorder, and/or impairment associated with the phenotypic profile of the respective entity.

Examples of inventive and technologically advantageous embodiments of the present disclosure include: (i) an ML model trained to predict and generate an individual risk score for an entity in a disparity group; (ii) an ML model configured to refine and/or adjust an individual risk score in order to prioritize said entity to address social and/or demographic inequalities; (iii) an ML model configured to generate and/or facilitate the execution of various prediction-based actions associated with an entity in a disparity group; and (iv) an ML model configured to initiate and/or facilitate the execution of one or more evaluations associated with an entity. Other technical improvements and advantages may be realized by one of ordinary skill in the art.

It should be appreciated that while specific examples given with regard to the various embodiments described herein may pertain to one or more clinical domains, medical domains, healthcare domains and/or insurance domains, persons of ordinary skill in the art will realize that the methods associated with the embodiments described herein could be effectively applied to one or more financial domains, engineering domains, aerospace domains, industrial domains, petrochemical domains, agricultural domains, educational domains, and/or any other relevant, complex scientific and/or technological domain.

### Example System Operations

As indicated, various embodiments of the present disclosure make important technical contributions to model-based risk prediction and pathway prioritization technology. In particular, systems and methods are disclosed herein that implement an ML-based, multi-modal approach to predicting and/or mitigating the individual risk of entities associated with a respective disparity group. Unlike traditional training techniques, the ML-based techniques of the present disclosure leverage at least one or more of a risk prediction model, a disparity risk adjustment model, an evaluation risk adjustment model, and/or a multi-dimensional location ranking model to generate individual risk scores, disparity adjusted risk scores, evaluation adjusted risk scores, prediction-based actions, prediction-based recommendations, and/or phenotypic profiles for one or more entities associated with the respective disparity group. As described herein, the various ML models associated with the risk prediction model may be leveraged to generate individual, prediction-based action sequences based on the individual risk scores and/or the phenotypic profile associated with the respective entity.

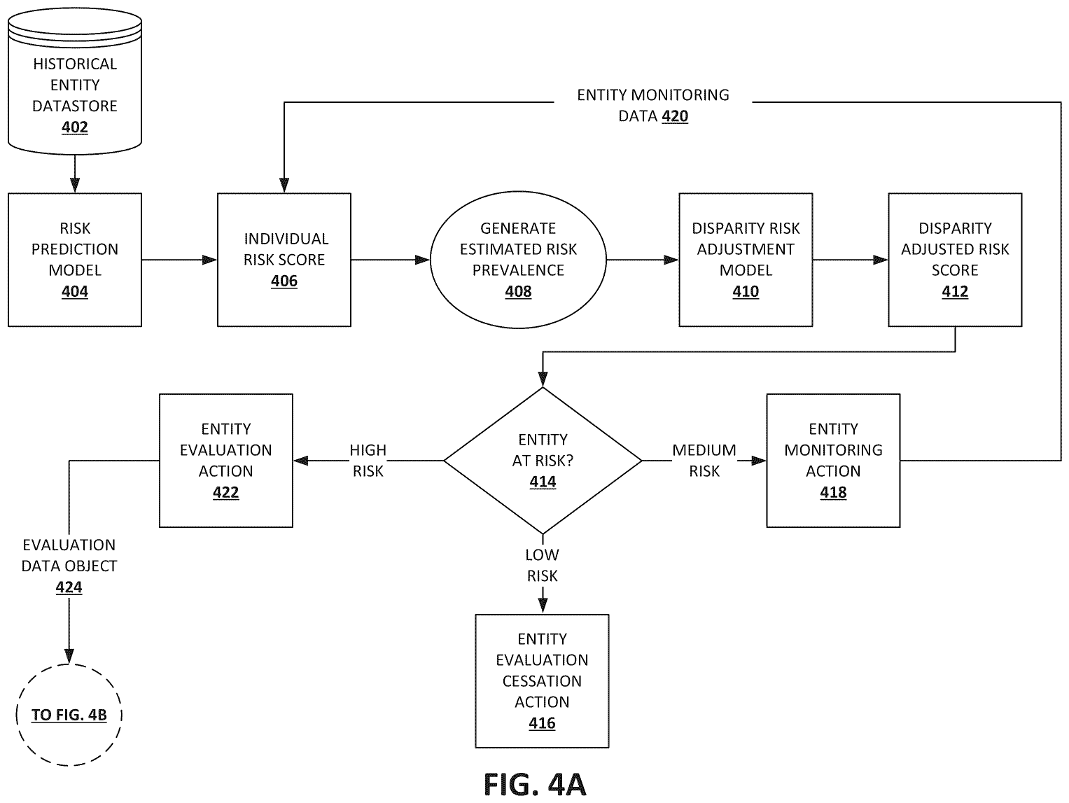

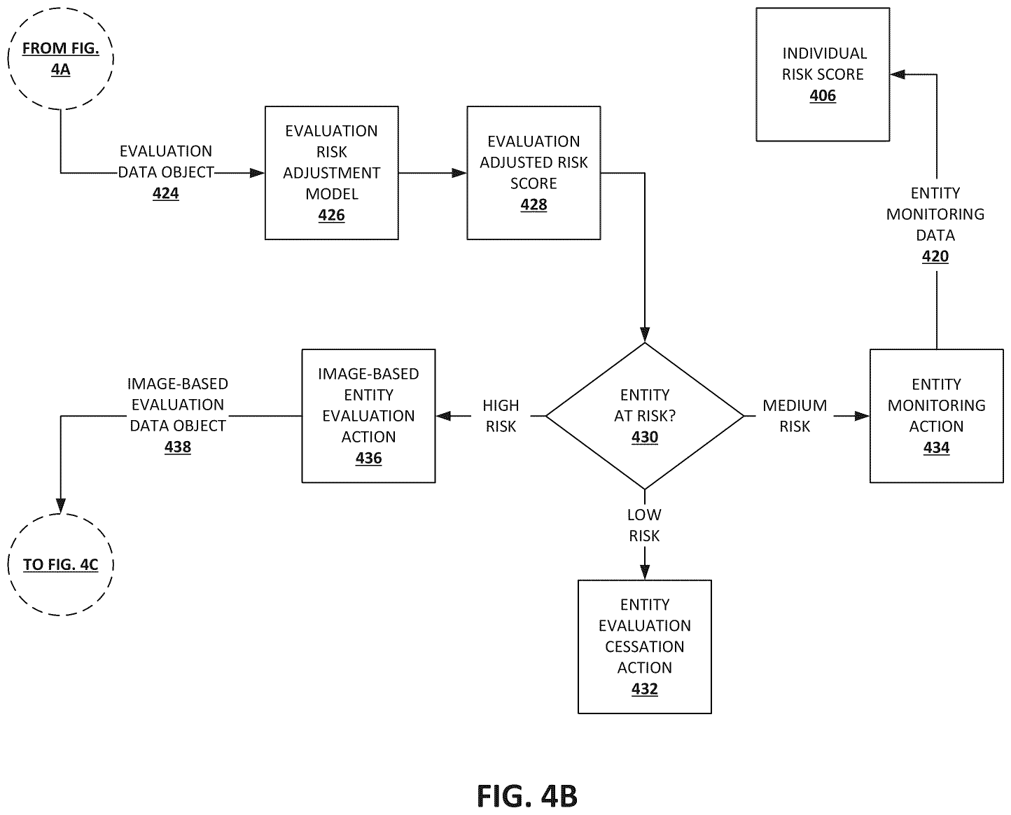

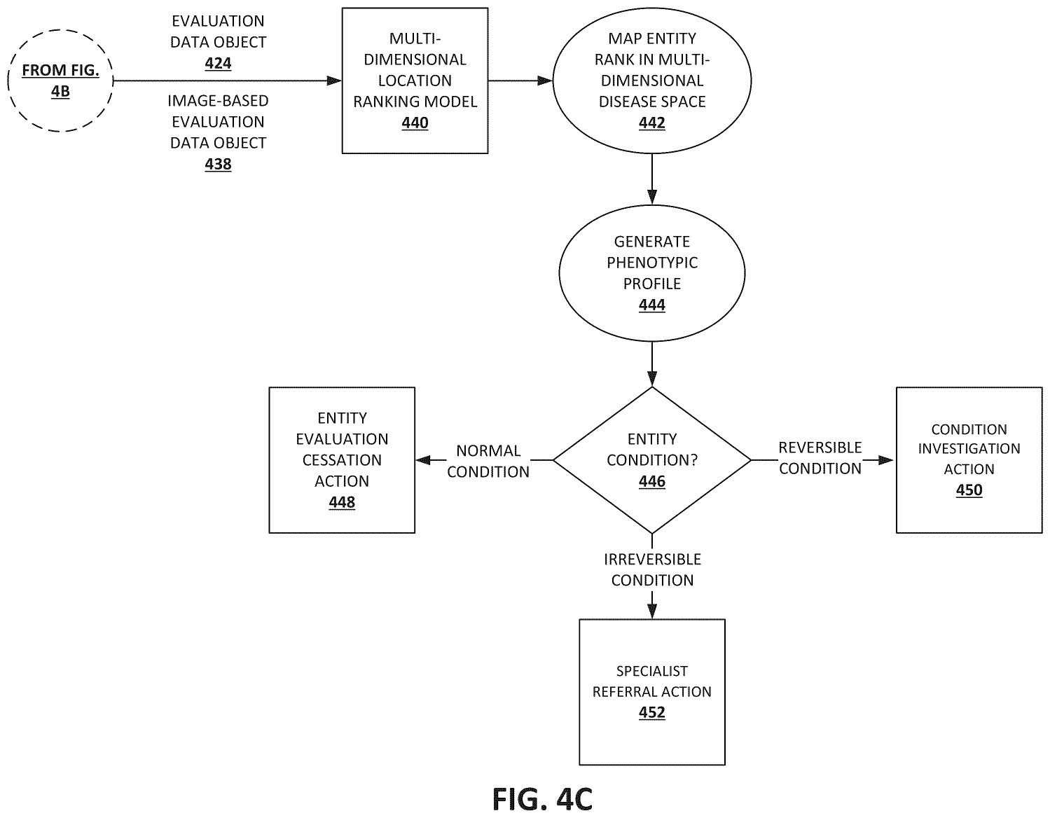

FIGS. 4A-C provide a dataflow diagram showing example data structures, modules, and operations for performing model-based risk prediction and treatment pathway prioritization in accordance with some embodiments discussed herein. For example, as shown in FIG. 4A, a risk prediction model 404 may configured to generate an individual risk score 406 for a respective entity associated with a particular disparity group. The individual risk score 406 may be generated based on one or more portions of data related to the entity comprised in a historical entity datastore 402. In various embodiments, the historical entity datastore 402 may be configured as an electronic medical record (EMR) datastore and the individual risk score 406 may be associated with a probability that a respective entity has, or will develop, experience, and/or undergo an adverse outcome, situation, and/or condition associated with one or more technical domains (e.g., healthcare domains, financial domains, insurance domains, and/or the like). For example, in the context of the healthcare domain, an individual risk score 406 may indicate if an entity is at a high, medium, or low risk of having and/or developing, a particular disease, disorder, and/or impairment, such as cognitive impairment, dementia, heart disease, diabetes, renal disease, and/or the like.

In some embodiments, an individual risk score 406 is generated by a risk prediction model 404 based on historical records (e.g., medical records) related to a respective entity stored in the historical entity datastore 402. For example, in a healthcare domain context, an individual risk score 406 may be configured to measure a likelihood of an individual entity having or developing a particular disease based on an EMR dataset associated with the entity stored in the historical entity datastore 402, where the EMR dataset comprises at least one or more of a personal medical history, a family medical history, a disease risk factor, a head trauma history and/or the like previously recorded for an individual entity. In some embodiments, a risk prediction model 404 may generate an individual risk score 406 for each entity of a disparity group associated with the historical entity datastore 402.

Operation 408 describes that, in various embodiments, the risk prediction model 404 may generate an estimated risk prevalence for a particular disparity group (e.g., an estimated dementia risk prevalence for the particular disparity group). In some embodiments, the estimated risk prevalence is determined based on aggregating the individual risk scores 406 for one or more respective entities in a disparity group associated with a respective entity cohort based on data comprised in the historical entity datastore 402. The estimated risk prevalence may be used by a disparity risk adjustment model 410 in order to generate a disparity adjusted risk score 412 for a respective entity associated with the particular disparity group.

For example, in some embodiments, the disparity adjusted risk score 412 associated with the respective entity is generated based on performing a disparity risk adjustment. The disparity risk adjustment may comprise generating, using the risk prediction model 404, a risk prevalence ratio, where the risk prevalence ratio is a ratio of a documented risk prevalence associated with a disparity group to the estimated risk prevalence associated with the disparity group generated in operation 408. As such, the disparity adjusted risk score 412 may be generated based on applying at least the risk prevalence ratio and an entity-defined disparity weighting parameter to the individual risk score 406 associated with the entity. Additionally or alternatively, in some embodiments, an individual risk score 406 associated with the respective entity may be updated based on entity monitoring data 420 generated by an entity monitoring computing device (e.g., a user computing entity 102a) associated with the respective entity.

In some embodiments, performing the disparity risk adjustment may comprise executing a disparity risk adjustment equation defined as:

$$P ( D ) = {e}^{log ( P ( D - 1 ) ) + ( β \times log ( 1 - R ) )} \qquad Equation 1$$

where P(D) is the disparity adjusted risk score 412 for the respective entity, P(D−1) is the individual risk score 406 for the respective entity, β is a user-defined disparity weighting parameter (e.g., having possible values are between 0 and 1), and R is a risk prevalence ratio, where the risk prevalence ratio is a ratio of a documented risk prevalence associated with the disparity group to an estimated risk prevalence associated with the disparity group. Generating disparity adjusted risk scores for respective entities has the effect of prioritizing entities in disparity groups where there is a greater level of undiagnosed adverse risks, situations, outcomes, and/or conditions (e.g., diseases, conditions, maladies, sicknesses, disorders, and/or impairments).

In some embodiments, a disparity adjusted risk score 412 may be used by a risk prediction model 404 to generate one or more recommendations and/or cause execution of one or more actions, instructions, and/or commands related to a respective entity. For example, in the healthcare domain, a disparity adjusted risk score 412 may be evaluated (e.g., via the risk prediction model 404) in comparison to one or more risk score thresholds to determine whether the respective entity is to be categorized as a high, medium, or low risk for a particular disease, condition, malady, sickness, disorder, and/or impairment. Based on the one or more risk score thresholds, the risk prediction model 404 may generate, receive, and/or manage one or more data objects related to the initiation and/or execution of one or more prediction-based actions, instructions, and/or commands related to the respective entity.

For example, at operation 414, the risk prediction model 404 may determine that the disparity adjusted risk score 412 associated with an entity satisfies a low risk score threshold and, as such, initiate, perform, and/or otherwise cause the execution of an entity evaluation cessation action 416. In some embodiments, the entity evaluation cessation action 416 is associated with a predetermined periodicity of time (e.g., 24 months) for which the entity is not further computationally evaluated with respect to the particular disease, condition, malady, sickness, disorder, and/or impairment related to the disparity adjusted risk score.

As another example, at operation 414, the risk prediction model 404 may determine that the disparity adjusted risk score 412 associated with an entity satisfies a medium risk score threshold and, as such, initiate, perform, and/or otherwise cause the execution of an entity monitoring action 418. In some embodiments, the entity monitoring action 418 is an employment of an entity monitoring computing device (e.g., a user computing entity 102a) to collect entity monitoring data 420 associated with the respective entity (e.g., data related to cognitive health, heart health, renal health, diabetic health, and/or the like). As such, in some embodiments, based on the entity monitoring action 418, the risk prediction model may cause association (e.g., issuance, delivery, network linkage, and/or monitoring) of an entity monitoring computing device with the respective entity. As described herein, in some embodiments, an individual risk score 406 associated with the respective entity may be updated based on entity monitoring data 420 generated by the entity monitoring computing device associated with the respective entity.

As another example, at operation 414, the risk prediction model 404 may determine that the disparity adjusted risk score 412 associated with an entity satisfies a high risk score threshold and, as such, initiate, perform, and/or otherwise cause the execution of an entity evaluation action 422. In some embodiments, the entity evaluation action 422 is associated with one or more physical, mental, and/or emotional evaluations related to the respective disease, condition, malady, sickness, disorder, and/or impairment associated with the disparity adjusted risk score 412. For example, in some embodiments, the entity evaluation action 422 is associated with a cognitive evaluation for the entity, where the cognitive evaluation is associated with at least one of a computer-scored and/or specialist-scored text-based entity evaluation (e.g., a handwriting evaluation, reading comprehension evaluation, memory evaluation, written evaluation, and/or the like), a speech-based entity evaluation (e.g., an evaluation to detect whether an entity is slurring, mispronouncing, and/or stuttering while speaking, and/or an interview-style evaluation administered to an entity), a gait-based entity evaluation (e.g., a walking evaluation, balance evaluation, standing evaluation, sitting evaluation, and/or the like), or a biomarker-based evaluation (e.g., a blood pressure evaluation, vascular evaluation, glucose evaluation, cerebral-spinal fluid evaluation, and/or the like). An evaluation data object 424 may be generated based on the entity evaluation action 422, and where the evaluation data object 424 may include data related to one or more evaluations of the entity (e.g., cognitive evaluation data).

In one or more embodiments, the risk prediction model 404 may generate an evaluation data object 424 associated with one or more portions of data associated with the entity evaluation action 422. For example, in some embodiments, the evaluation data object 424 may include cognitive evaluation scores, results, and/or other data generated based on a cognitive evaluation administered to the respective entity. Furthermore, in some embodiments, the risk prediction model 404 may generate correspondence associated with the entity evaluation action 422 (e.g., informational correspondence, scheduling correspondence, insurance correspondence, etc.) and cause transmission, delivery, notification, and/or data indicative of (e.g., including one or more evaluation identifiers, etc.) the correspondence associated with the entity evaluation action 422 to the respective entity.

Referencing FIG. 4B, an evaluation risk adjustment model 426 may receive, retrieve, and/or otherwise process the evaluation data object 424 to generate an evaluation adjusted risk score 428. In some embodiments, the evaluation adjusted risk score 428 associated with the respective entity is generated based on performing an evaluation risk adjustment. For example, in the healthcare domain, the evaluation risk adjustment may be performed by the evaluation risk adjustment model 426 in response to determining that a disparity adjusted risk score 412 associated with a respective entity satisfies a high risk score threshold indicating that the respective entity is at a high risk for a particular disease, condition, malady, sickness, disorder, and/or impairment.

The evaluation risk adjustment model 426 may generate the evaluation adjusted risk score 428 for the entity based on the disparity adjusted risk score 412 and an evaluation data object 424 associated with the entity, where the evaluation data object 424 is generated based on the entity evaluation action 422, and where the evaluation data object 424 may include data related to one or more evaluations of the entity (e.g., cognitive evaluation scores, results, and/or other data). In some embodiments, the evaluation data comprised in an evaluation data object 424 associated with a respective entity may be compared to normative median evaluation values (e.g., median evaluation values associated with entities that do not, or will not, have the particular disease, condition, malady, sickness, disorder, and/or impairment the respective entity is at risk for).

In some embodiments, the evaluation data object 424 may include cognitive evaluation scores associated with the respective entity, and the cognitive evaluation scores may be divided by a normative median cognitive evaluation score. As such, the resulting cognitive evaluation scores may be employed in the evaluation risk adjustment in order to prioritize entities associated with a disparity group and/or entity cohort with lower cognitive evaluation scores for subsequent prediction-based actions (e.g., an image-based entity evaluation action). For example, in some embodiments, the risk prediction model 404 may employ a variation of Equation 1 in which the value for R is associated with a ratio of the cognitive evaluation scores of the respective entity to the normative median cognitive evaluation score in order to generate an evaluation adjusted risk score 428 for a respective entity. Additionally or alternatively, in some embodiments, the risk prediction model 404 may employ a variation of Equation 1 in which the value for R is associated with a ratio of entity monitoring data associated with the respective entity (e.g., blood pressure data generated by an entity monitoring computing device) to documented normative median monitoring data (e.g., a normative median blood pressure range associated with a particular entity cohort, population, and/or group) in order to generate the evaluation adjusted risk score 428 for a respective entity.

In some embodiments, the evaluation adjusted risk score 428 may be compared to one or more risk score thresholds to determine whether to generate one or more subsequent prediction-based actions associated with the entity. For example, in some embodiments, an evaluation adjusted risk score 428 may be used by a risk prediction model 404 to generate, receive, and/or manage one or more data objects related to the initiation and/or execution of one or more prediction-based actions, instructions, and/or commands related to the respective entity (e.g., a respective medical patient). For example, an evaluation adjusted risk score 428 may be evaluated (e.g., via the risk prediction model 404) in comparison to one or more risk score thresholds to determine whether the respective entity is to be categorized as a high, medium, or low risk for developing, experiencing, and/or undergoing an adverse outcome, situation, and/or condition associated with one or more technical domains (e.g., healthcare domains, financial domains, insurance domains, and/or the like). For example, in the context of the healthcare domain, an evaluation adjusted risk score 428 may indicate if an entity is at a high, medium, or low risk of having and/or developing, a particular disease, disorder, and/or impairment, such as cognitive impairment, dementia, heart disease, diabetes, renal disease, and/or the like. Based on the one or more risk score thresholds, the risk prediction model 404 may generate, receive, and/or manage one or more data objects related to the initiation and/or execution of one or more prediction-based actions, instructions, and/or commands related to the respective entity.

For example, at operation 430, the risk prediction model 404 may determine that the evaluation adjusted risk score 428 associated with an entity satisfies a low risk score threshold and, as such, initiate, perform, and/or otherwise cause the execution of an entity evaluation cessation action 432. In some embodiments, the entity evaluation cessation action 432 is associated with a predetermined periodicity of time (e.g., 24 months) for which the entity is not further computationally evaluated with respect to the particular disease, condition, malady, sickness, disorder, and/or impairment related to the disparity adjusted risk score.

As another example, at operation 430, the risk prediction model 404 may determine that the evaluation adjusted risk score 428 associated with an entity satisfies a medium risk score threshold and, as such, initiate, perform, and/or otherwise cause the execution of an entity monitoring action 434. In some embodiments, the entity monitoring action 434 is an employment of an entity monitoring computing device (e.g., a user computing entity 102a) to collect entity monitoring data 420 associated with the respective entity (e.g., data related to cognitive health, heart health, renal health, diabetic health, and/or the like). As such, in some embodiments, based on the entity monitoring action 434, the risk prediction model may cause association (e.g., issuance, delivery, network linkage, and/or monitoring) of an entity monitoring computing device with the respective entity. As described herein, in some embodiments, an individual risk score 406 associated with the respective entity may be updated based on entity monitoring data 420 generated by the entity monitoring computing device associated with the respective entity.

As another example, at operation 430, the risk prediction model 404 may determine that the evaluation adjusted risk score 428 associated with an entity satisfies a high risk score threshold and, as such, initiate, perform, and/or otherwise cause the execution of an image-based entity evaluation action 436. In some embodiments, an image-based entity evaluation action 436 may be an imaging action such as a magnetic resonance imaging (MRI) procedure, a fluid attenuated inversion recovery (FLAIR) sequence, positron emission tomography (PET) procedure, or computed tomography (CT) procedure performed with respect to one or more body parts of an entity. In some embodiments, the risk prediction model 404 is configured to generate, receive, retrieve, and/or otherwise process an image-based evaluation data object 438 associated with an image-based entity evaluation action 436. In some embodiments, an image-based evaluation data object 438 may comprise one or more portions of imaging data related to one or more imaging procedures performed with reference to one or more body parts of a respective entity.

Referencing FIG. 4C, a multi-dimensional location ranking model 440 may be configured to receive, retrieve, manage, and/or otherwise process an evaluation data object 424 and/or an image-based evaluation data object 438. With reference to operations 442 and 444, in some embodiments, the multi-dimensional location ranking model 440 may be configured to perform multi-dimensional location ranking via Euclidean distance in order to facilitate the mapping of an entity rank associated with a respective entity in a multi-dimensional disease space. By mapping the entity rank in the multi-dimensional disease space, the multi-dimensional location ranking model 440 may generate a phenotypic profile for the respective entity. In some embodiments, a phenotypic profile comprises at least one of a disease severity and/or a disease subtype associated with the respective entity. The disease severity and/or the disease subtype may characterize, describe, and/or indicate one or more attributes related to a respective disease, condition, malady, sickness, disorder, and/or impairment associated with the respective entity. Furthermore, the phenotypic profile may describe whether a current condition of the respective entity is a normal condition, reversible condition, or irreversible condition.

In some embodiments, median normative evaluation data and median image-based evaluation data provide the basis for multi-dimensional location ranking via Euclidean distance. For example, the evaluation data comprised in an evaluation data object 424 and/or image data comprised in an image-based evaluation data object 438 associated with a respective entity may be compared to normative median evaluation values (e.g., median evaluation values associated with entities that do not, or will not, have the particular disease, condition, malady, sickness, disorder, and/or impairment the respective entity is at risk for).

In some examples, while the evaluation data comprised in an evaluation data object 424 may already be in a numerical form, the image data comprised in an image-based evaluation data object 438 (e.g., brain MRI results rendered in a 3-dimensional image format) may require additional preprocessing before the image data may be employed in multi-dimensional location ranking. In examples related to the cognitive health domain, brain tissue and lobar segmentation may be performed to transform brain image data related to a respective entity into a numerical form. In such examples, the proportion of brain tissue (e.g., gray matter and white matter) per brain lobe (e.g., frontal, parietal, temporal, and/or occipital lobes) may be calculated on an individual entity basis and groupwise median values may be obtained from a normative population. In some embodiments, if PET is performed on a respective entity, average PET centiloid values may also be computed per lobe.

In some embodiments, the multi-dimensional location ranking model 440 may be configured to perform multi-dimensional location ranking via Euclidean distance for a plurality of domain data (e.g., imaging, cognition, blood, and/or other biomarker data that is clinically indicated) via a multi-dimensional location ranking equation, defined as:

$$r = \sqrt{{( {i}_{n} - {i}_{p} )}^{2} + … + {( {C}_{n} - {C}_{p} )}^{2}} \qquad Equation 2$$

where r is an entity rank associated with a distance from a multi-dimensional normative median, iₙ is a normative median imaging value, iₚ is an imaging value associated with a particular entity (e.g., imaging data associated with an image-based evaluation data object 438), Cₙ is a normative median cognition value, and Cₚ is an entity cognition evaluation value (e.g., cognitive evaluation data associated with an evaluation data object 424). The coefficients i and C may be single numbers or vectors corresponding to multiple evaluations within each respective domain. In some embodiments, additional domains (e.g., indicated by “ . . . ”) may be added to the multi-dimensional location ranking equation in the same form as presented above for i and C, (e.g., an addition of the square of entity values subtracted from normative median values).

The value for r may provide a degree of disease severity and/or location in a multi-dimensional disease space. Each of the individual domains associated with the coefficients considered in the multi-dimensional location ranking equation (e.g., i and C) may be associated with the multi-dimensional disease space such that an assessment of a distance value (e.g., Euclidian distance value) of each individual domain may be employed by the multi-dimensional location ranking model 440 to determine a phenotypic profile for a respective entity. For example, in the cognitive health domain, the values for r, i, and C may suggest a particular dementia subtype (e.g., a large value for r coupled with long distance values for cognitive behavioral problems and frontal and/or temporal brain tissue loss (e.g., as evidenced by image data) may indicate a behavioral variant frontotemporal dementia phenotypic profile for a respective entity.

At operation 446, the risk prediction model 404 may be configured to generate, receive, and/or manage one or more data objects related to the generating, initiation, and/or execution of one or more prediction-based action sequences, prediction-based actions, instructions, and/or commands related to the respective entity based on data associated with the phenotypic profile generated for the respective entity. For example, at operation 446, the risk prediction model 404 may determine that a phenotypic profile indicates that a condition associated with a respective entity is a normal condition. As such, the risk prediction model 404 may generate, initiate, perform, and/or otherwise cause the execution of a prediction-based action sequence comprising an entity evaluation cessation action 448. In some embodiments, the entity evaluation cessation action 448 is associated with a predetermined periodicity of time (e.g., 24 months) for which the entity is not further computationally evaluated with respect to the particular disease, condition, malady, sickness, disorder, and/or impairment described by the phenotypic profile associated with the respective entity.

As another example, at operation 446, the risk prediction model 404 may determine that a phenotypic profile indicates that a condition associated with a respective entity is a reversible condition. As such, the risk prediction model 404 may generate, initiate, perform, and/or otherwise cause the execution of a prediction-based action sequence comprising a condition investigation action 450. In some embodiments, the condition investigation action 450 is associated with a referral, an additional evaluation, additional research, and/or additional analysis associated with the respective entity and/or the particular disease, condition, malady, sickness, disorder, and/or impairment associated with the respective entity. For example, in the cognitive health domain, entities with potentially reversible cognitive impairment (e.g., entities without atrophy, small vessel disease, and/or other biomarker involvement) may be referred for additional evaluations that are tangentially related to a current condition of the entity (e.g., evaluations associated with mental disorders (e.g., clinical depression), delirium, hydrocephalus and/or the like).

As another example, at operation 446, the risk prediction model 404 may determine that a phenotypic profile indicates that a condition associated with a respective entity is an irreversible condition. As such, the risk prediction model 404 may generate, initiate, perform, and/or otherwise cause the execution of a prediction-based action sequence comprising a specialist referral action 452 associated with the respective entity. In such examples, the risk prediction model 404 may generate a specialist referral associated with a respective medical specialist based on the specialist referral action 452. Furthermore, in some embodiments, the risk prediction model 404 may generate correspondence associated with the specialist referral (e.g., informational correspondence, scheduling correspondence, insurance correspondence, etc.) and cause transmission, delivery, notification, and/or data indicative of (e.g., including one or more referral identifiers, etc.) the correspondence associated with the specialist referral to the respective entity. For example, in the cognitive health domain, entities with irreversible cognitive impairment (e.g., entities with severe brain tissue atrophy and/or a large amyloid plaque burden) may be referred to a relevant neurologist, old-age psychiatrist, and/or care facility.

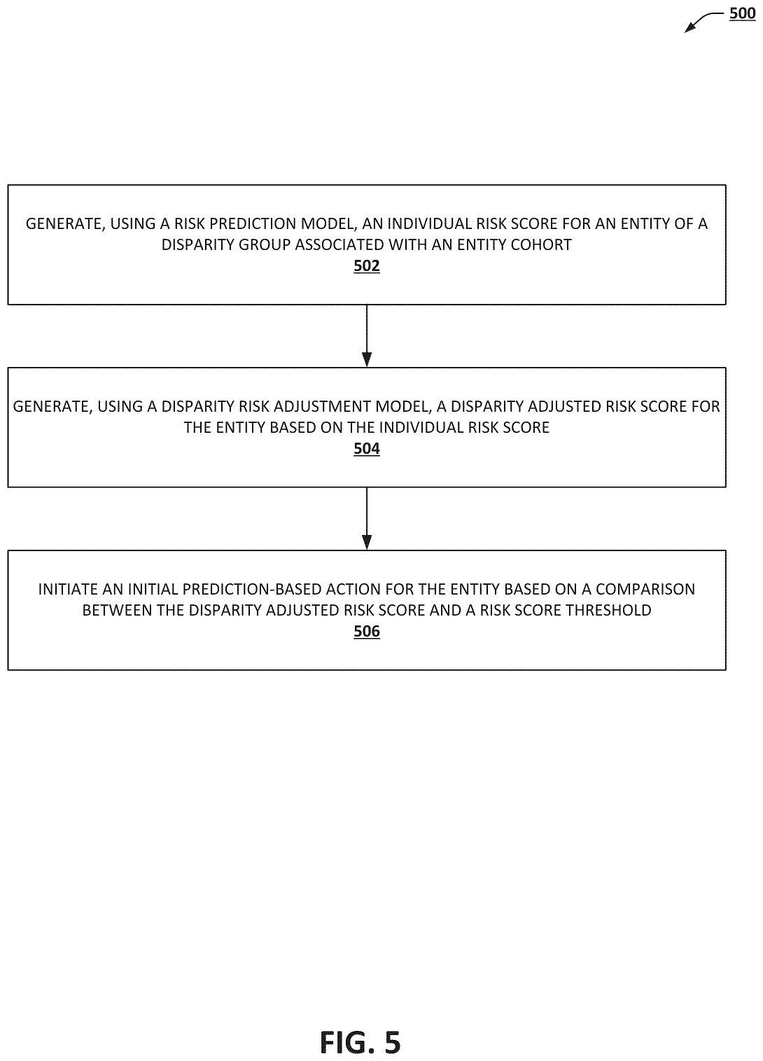

FIG. 5 is a flowchart showing an example of a process 500 for initiating a prediction-based action based on a disparity adjusted risk score generated for a respective entity in accordance with some embodiments discussed herein. The flowchart depicts the generation and refinement of an individual risk score associated with an entity related to a respective disparity group. The generation and refinement of the individual risk score may be implemented by one or more computing devices, entities, and/or systems described herein. For example, via the various steps/operations of the process 500, the computing system 100 may leverage a risk prediction model and/or a disparity risk adjustment model to overcome the various limitations associated with traditional risk profiling techniques by initiating a prediction-based action for the entity based on the disparity adjusted risk score.

FIG. 5 illustrates an example process 500 for explanatory purposes. Although the example process 500 depicts a particular sequence of steps/operations, the sequence may be altered without departing from the scope of the present disclosure. For example, some of the steps/operations depicted may be performed in parallel or in a different sequence that does not materially impact the function of the process 500. In other examples, different components of an example device or system that implements the process 500 may perform functions at substantially the same time or in a specific sequence.

In some embodiments, the process 500 includes, at step/operation 502, generating an individual risk score for an entity. For example, the computing system 100 may generate, using a risk prediction model 404, an individual risk score 406 for an entity of a disparity group associated with an entity cohort. In some embodiments, a disparity group may include a subset of a plurality of entities within the particular entity cohort that are grouped based on one or more criteria. By way of example, a disparity group may include a subset of entities that share one or more common and/or related attributes that are defined by one or more criteria. For example, a disparity group may be associated with a particular geographic region. Additionally or alternatively, a disparity group may be associated with one or more contextual attributes. The one or more contextual attributes may be associated with one or more demographic characteristics (e.g., race, ethnicity, gender, age, lifestyle choices, and/or the like), membership plans (e.g., healthcare plan, insurance plan, etc.), and/or may describe one or more common entity experiences (e.g., access to healthcare, access to doctors, access to specialists, access to transportation, financial needs, and/or the like).

In some embodiments, the process 500 includes, at step/operation 504, generating a disparity adjusted risk score for an entity. For example, the computing system 100 may generate, using a disparity risk adjustment model 410, a disparity adjusted risk score 412 for the entity based on the individual risk score 406. In this regard, the risk prediction model 404 may be configured to perform a disparity risk adjustment, where the disparity risk adjustment comprises generating, using the risk prediction model 404, a risk prevalence ratio, where the risk prevalence ratio is a ratio of a documented risk prevalence associated with the disparity group to an estimated risk prevalence associated with the disparity group. In some embodiments, the estimated risk prevalence is determined based on aggregating the individual risk score 406 for one or more respective entities in the disparity group associated with the entity cohort. As such, the disparity risk adjustment model 410 may generate the disparity adjusted risk score 412 based on applying at least the risk prevalence ratio and an entity-defined disparity weighting parameter to the individual risk score 406 associated with the entity. As described herein, in some embodiments, the estimated risk prevalence associated with the disparity group is updated based on the disparity adjusted risk score 412 associated with one or more entities.

In some embodiments, the process 500 includes, at step/operation 506, initiating an initial prediction-based action for the entity. For example, the computing system 100 may initiate an initial prediction-based action for the entity based on a comparison between the disparity adjusted risk score 412 and a risk score threshold. In some examples, the risk score threshold may be a medium risk score threshold and, accordingly, the initial prediction-based action may be an entity monitoring action 418. In such examples, the risk prediction model 404 may be configured to initiate, based on the entity monitoring action 418, association of an entity monitoring computing device (e.g., a user computing entity 102a) to the entity. Furthermore, in such examples, the risk prediction model 404 may be configured to receive entity monitoring data 420 associated with the entity, where the entity monitoring data 420 is generated by the entity monitoring computing device. As such, the risk prediction model 404 may be configured to update the individual risk score 406 associated with the entity based on the entity monitoring data 420.

Alternatively, in some examples, the risk score threshold may be a low risk score threshold and, accordingly, the initial prediction-based action may be an entity evaluation cessation action 416. In some embodiments, the entity evaluation cessation action 416 is associated with a predetermined periodicity of time (e.g., 24 months) for which the entity is not further computationally evaluated with respect to the particular disease, condition, malady, sickness, disorder, and/or impairment related to the disparity adjusted risk score. Alternatively, in some examples, the risk score threshold may be a high risk score threshold and, accordingly, the initial prediction-based action may be an entity evaluation action 422.

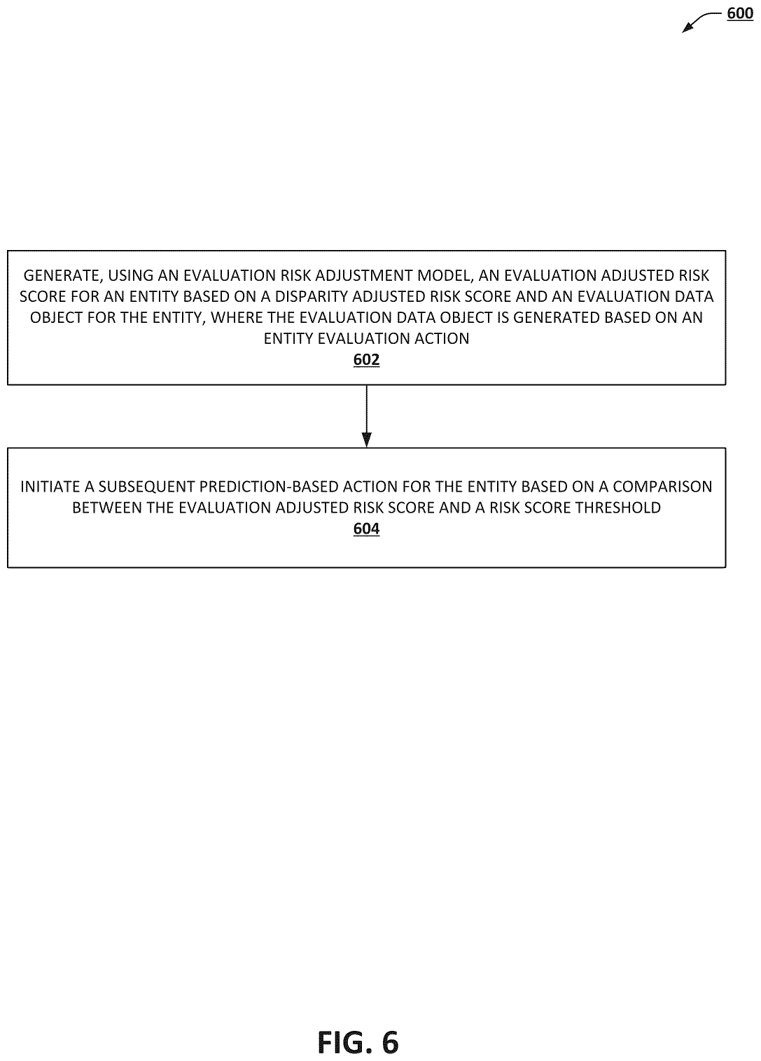

FIG. 6 is a flowchart showing an example of a process for initiating a subsequent prediction-based action based on an evaluation adjusted risk score generated for a respective entity in accordance with some embodiments discussed herein. The flowchart depicts the generation of an evaluation adjusted risk score associated with an entity related to a respective disparity group. The generation of an evaluation adjusted risk score and the initiation a subsequent prediction-based action may be implemented by one or more computing devices, entities, and/or systems described herein. For example, via the various steps/operations of the process 600, the computing system 100 may generate and leverage an evaluation risk adjustment model to overcome the various limitations associated with traditional risk profiling techniques by initiating a subsequent prediction-based action for the entity based on the evaluation adjusted risk score.

FIG. 6 illustrates an example process 600 for explanatory purposes. Although the example process 600 depicts a particular sequence of steps/operations, the sequence may be altered without departing from the scope of the present disclosure. For example, some of the steps/operations depicted may be performed in parallel or in a different sequence that does not materially impact the function of the process 600. In other examples, different components of an example device or system that implements the process 600 may perform functions at substantially the same time or in a specific sequence.

In some embodiments, the process 600 includes, at step/operation 602, generating an evaluation adjusted risk score 428 for an entity. For example, the computing system 100 may generate, using an evaluation risk adjustment model 426, an evaluation adjusted risk score 428 for an entity based on a disparity adjusted risk score 412 and an evaluation data object 424 for the entity, where the evaluation data object 424 is generated based on an entity evaluation action 422. In some embodiments, the entity evaluation action 422 is associated with a cognitive evaluation for the entity, and where the cognitive evaluation is associated with at least one of a computer-scored and/or specialist-scored text-based entity evaluation (e.g., a handwriting evaluation, reading comprehension evaluation, memory evaluation, written evaluation, and/or the like), a speech-based entity evaluation (e.g., an evaluation to detect whether an entity is slurring, mispronouncing, and/or stuttering while speaking, and/or an interview-style evaluation administered to an entity), a gait-based entity evaluation (e.g., a walking evaluation, balance evaluation, standing evaluation, sitting evaluation, and/or the like), or a biomarker-based evaluation (e.g., a blood pressure evaluation, vascular evaluation, glucose evaluation, cerebral-spinal fluid evaluation, and/or the like). In some embodiments, the risk prediction model 404 may generate correspondence associated with the entity evaluation action 422 (e.g., informational correspondence, scheduling correspondence, insurance correspondence, etc.) and cause transmission, delivery, notification, and/or data indicative of (e.g., including one or more evaluation identifiers, etc.) the correspondence associated with the entity evaluation action 422 to the respective entity.

In some embodiments, the process 600 includes, at step/operation 604, initiating a subsequent prediction-based action for the entity. For example, the computing system 100 may initiate a subsequent prediction-based action for the entity based on a comparison between the evaluation adjusted risk score 428 and a risk score threshold. In some examples, the risk score threshold may be a high risk score threshold and, accordingly, the subsequent prediction-based action may be an image-based entity evaluation action 436. In some embodiments, an image-based entity evaluation action 436 may be an imaging action such as an MRI procedure, a FLAIR sequence, PET procedure, or CT procedure performed with respect to one or more body parts of an entity. In some embodiments, the risk prediction model 404 is configured to generate, receive, retrieve, and/or otherwise process an image-based evaluation data object 438 associated with an image-based entity evaluation action 436. In some embodiments, an image-based evaluation data object 438 may comprise one or more portions of imaging data related to one or more imaging procedures performed with reference to one or more body parts of a respective entity.

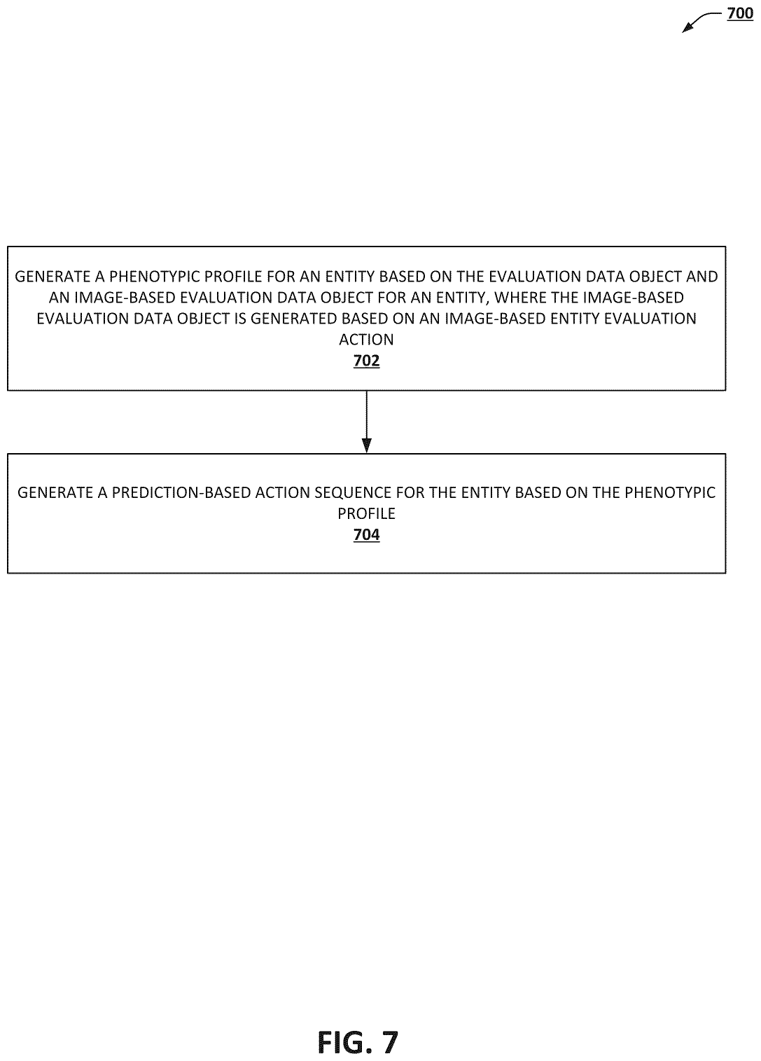

FIG. 7 is a flowchart showing an example of a process for generating a prediction-based action sequence for a respective entity based on a phenotypic profile generated for the respective entity in accordance with some embodiments discussed herein. The flowchart depicts the generation of a phenotypic profile for an entity related to a respective disparity group. The generation of a phenotypic profile and the subsequent generation of a prediction-based action sequence may be implemented by one or more computing devices, entities, and/or systems described herein. For example, via the various steps/operations of the process 700, the computing system 100 may generate and leverage a risk prediction model to overcome the various limitations associated with traditional risk profiling techniques by generating a prediction-based action sequence for a respective entity based on a phenotypic profile generated for the respective entity.

FIG. 7 illustrates an example process 700 for explanatory purposes. Although the example process 700 depicts a particular sequence of steps/operations, the sequence may be altered without departing from the scope of the present disclosure. For example, some of the steps/operations depicted may be performed in parallel or in a different sequence that does not materially impact the function of the process 700. In other examples, different components of an example device or system that implements the process 700 may perform functions at substantially the same time or in a specific sequence.

In some embodiments, the process 700 includes, at step/operation 702, generating a phenotypic profile for an entity. For example, the computing system 100 may generate a phenotypic profile for an entity based on an evaluation data object 424 and an image-based evaluation data object 438 for an entity, where the image-based evaluation data object 438 is generated based on an image-based entity evaluation action 436. In some embodiments, generating a phenotypic profile for an entity may comprise generating, using a multi-dimensional location ranking model 440, an entity rank associated with the entity based on the evaluation data object 424 and the image-based evaluation data object 438. The multi-dimensional location ranking model 440 may subsequently map the entity rank in a multi-dimensional disease space associated with a plurality of phenotypic profiles in order to generate the phenotypic profile associated with the entity. In some embodiments, a phenotypic profile comprises at least one of a disease severity and/or a disease subtype associated with the respective entity. The disease severity and/or the disease subtype may characterize, describe, and/or indicate one or more attributes related to a respective disease, condition, malady, sickness, disorder, and/or impairment associated with the respective entity. Furthermore, the phenotypic profile may describe whether a current condition of the respective entity is a normal condition, reversible condition, or irreversible condition.

In some embodiments, the process 700 includes, at step/operation 704, generating a prediction-based action sequence for the respective entity. For example, the computing system 100 may generate a prediction-based action sequence for the entity based on the phenotypic profile. In some examples, the prediction-based action sequence for the entity may be at least one of an entity evaluation cessation action 448, a condition investigation action 450, and/or a specialist referral action 452.

Some techniques of the present disclosure enable the generation of action outputs that may be performed to initiate one or more predictive actions to achieve real-world effects. The model-based risk prediction and treatment pathway prioritization techniques of the present disclosure may be used, applied, and/or otherwise leveraged to generate individual risk scores for entities of a disparity group, which may help in the computer-based risk profiling of the entities. The risk prediction model of the present disclosure may be leveraged to initiate the performance of various computing tasks that improve the performance of a computing system (e.g., a computer itself, etc.) with respect to various predictive actions performed by the computing system 100, such as for the generation of various data objects related to one or more prediction-based based actions associated with one or more entities.

Example predictive actions may include the generation of individual risk scores, disparity adjusted risk scores, evaluation adjusted risk scores, recommendations, and/or phenotypic profiles for one or more entities associated with a disparity group, entity cohort, and/or historical entity datastore. The risk prediction model may also be configured to initiate and/or cause execution of one or more prediction-based actions for one or more respective entities based on a comparison of one or more of a disparity adjusted risk score and/or an evaluation adjusted risk score associated with a respective entity to one or more risk score thresholds. In this regard, the risk prediction model may initiate and/or facilitate the execution of one or more entity evaluation actions, image-based entity evaluation actions, entity monitoring actions, and/or entity evaluation cessation actions.

In some examples, the computing tasks may include predictive actions that may be based on a prediction domain. A prediction domain may include any environment in which computing systems may be applied to achieve real-word insights and initiate the performance of computing tasks, such as entity evaluation actions and/or entity monitoring actions (e.g., to gather current health data related to an entity) to act on the real-world insights. In various examples, these real-world insights are leveraged to address regional and/or demographic inequalities related to various domains (e.g., healthcare domains, financial domains, and/or the like). These predictive actions may cause real-world changes, for example, by controlling a hardware component, providing alerts, interactive actions, and/or the like to mitigate the aforementioned regional and/or demographic inequalities.

### CONCLUSION

Many modifications and other embodiments will come to mind to one skilled in the art to which the present disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

### Examples

Example 1. A computer-implemented method, the computer-implemented method comprising generating, by one or more processors and using a risk prediction model, an individual risk score for an entity of a disparity group associated with an entity cohort; generating, by the one or more processors and using a disparity risk adjustment model, a disparity adjusted risk score for the entity based on the individual risk score; and initiating, by the one or more processors, an initial prediction-based action for the entity based on a comparison between the disparity adjusted risk score and a risk score threshold.

Example 2. The computer-implemented method of example 1, wherein the risk score threshold is a high risk score threshold, the initial prediction-based action is an entity evaluation action, and the computer-implemented method further comprises generating, using an evaluation risk adjustment model, an evaluation adjusted risk score for the entity based on the disparity adjusted risk score and an evaluation data object for the entity, wherein the evaluation data object is generated based on the entity evaluation action; and initiating, by the one or more processors, a subsequent prediction-based action for the entity based on a comparison between the evaluation adjusted risk score and the risk score threshold.

Example 3. The computer-implemented method of example 2, wherein the entity evaluation action is associated with a cognitive evaluation for the entity, and wherein the cognitive evaluation is associated with at least one of a text-based entity evaluation, a speech-based entity evaluation, a gait-based entity evaluation, or a biomarker-based evaluation.

Example 4. The computer-implemented method of any of examples 2 or 3, wherein the computer-implemented method further comprises generating, by the one or more processors and using the risk prediction model, correspondence associated with the entity evaluation action; and

providing, by the one or more processors, data indicative of the correspondence associated with the entity evaluation action.

Example 5. The computer-implemented method of any of examples 2 to 4, wherein the subsequent prediction-based action is an image-based entity evaluation action and the computer-implemented method further comprises generating, by the one or more processors, a phenotypic profile for the entity based on the evaluation data object and an image-based evaluation data object for the entity, wherein the image-based evaluation data object is generated based on the image-based entity evaluation action; and generating, by the one or more processors, a prediction-based action sequence for the entity based on the phenotypic profile.

Example 6. The computer-implemented method of example 5, wherein generating the phenotypic profile further comprises generating, by the one or more processors and using a multi-dimensional location ranking model, an entity rank associated with the entity based on the evaluation data object and the image-based evaluation data object; and mapping, by the one or more processors, the entity rank in a multi-dimensional disease space associated with a plurality of phenotypic profiles.

Example 7. The computer-implemented method of any of examples 5 or 6, wherein the phenotypic profile associated with the entity comprises at least one of a disease severity or a disease subtype and the phenotypic profile describes whether a current condition of the entity is a normal condition, reversible condition, or irreversible condition.

Example 8. The computer-implemented method of any of examples 5 to 7, wherein the computer-implemented method further comprises generating, by the one or more processors and using the risk prediction model, a specialist referral associated with a respective medical specialist based on the prediction-based action sequence; and providing, by the one or more processors, data indicative of the specialist referral to the entity.

Example 9. The computer-implemented method of any of the preceding examples, wherein generating the disparity adjusted risk score further comprises performing, by the one or more processors, a disparity risk adjustment, wherein the disparity risk adjustment comprises generating, using the risk prediction model, a risk prevalence ratio, wherein the risk prevalence ratio is a ratio of a documented risk prevalence associated with the disparity group to an estimated risk prevalence associated with the disparity group, wherein the estimated risk prevalence is determined based on aggregating the individual risk score for one or more respective entities in the disparity group associated with the entity cohort; and generating the disparity adjusted risk score based on applying at least the risk prevalence ratio and an entity-defined disparity weighting parameter to the individual risk score associated with the entity.

Example 10. The computer-implemented method of example 9, wherein the estimated risk prevalence associated with the disparity group is updated based on the disparity adjusted risk score associated with the entity.

Example 11. The computer-implemented method of any of the preceding examples, wherein the disparity group is associated with a geographic region and the disparity group is associated with one or more contextual attributes.

Example 12. The computer-implemented method of example 1, wherein the risk score threshold is a medium risk score threshold and the initial prediction-based action is an entity monitoring action.

Example 13. The computer-implemented method of example 12, wherein the computer-implemented method further comprises initiating, by the one or more processors based on the entity monitoring action, association of an entity monitoring computing device to the entity.

Example 14. The computer-implemented method of example 13, wherein the computer-implemented method further comprises receiving, by the one or more processors, entity monitoring data associated with the entity, wherein the entity monitoring data is generated by the entity monitoring computing device; and updating, by the one or more processors, the individual risk score associated with the entity based on the entity monitoring data.

Example 15. The computer-implemented method of example 1, wherein the risk score threshold is a low risk score threshold and the initial prediction-based action is an entity evaluation cessation action.

Example 16. A computing system comprising memory and one or more processors communicatively coupled to the memory, the one or more processors configured to generate, using a risk prediction model, an individual risk score for an entity of a disparity group associated with an entity cohort; generate, using a disparity risk adjustment model, a disparity adjusted risk score for the entity based on the individual risk score; and initiate an initial prediction-based action for the entity based on a comparison between the disparity adjusted risk score and a risk score threshold.

Example 17. The computing system of example 16, wherein the risk score threshold is a high risk score threshold, the initial prediction-based action is an entity evaluation action, and the one or more processors are further configured to generate, using an evaluation risk adjustment model, an evaluation adjusted risk score for the entity based on the disparity adjusted risk score and an evaluation data object for the entity, wherein the evaluation data object is generated based on the entity evaluation action; and initiate a subsequent prediction-based action for the entity based on a comparison between the evaluation adjusted risk score and the risk score threshold.

Example 18. The computing system of example 17, wherein the subsequent prediction-based action is an image-based entity evaluation action and the one or more processors are further configured to generate a phenotypic profile for the entity based on the evaluation data object and an image-based evaluation data object for the entity, wherein the image-based evaluation data object is generated based on the image-based entity evaluation action; and generate a prediction-based action sequence for the entity based on the phenotypic profile.

Example 19. One or more non-transitory computer-readable storage media including instructions that, when executed by one or more processors, cause the one or more processors to generate, using a risk prediction model, an individual risk score for an entity of a disparity group associated with an entity cohort; generate, using a disparity risk adjustment model, a disparity adjusted risk score for the entity based on the individual risk score; and initiate an initial prediction-based action for the entity based on a comparison between the disparity adjusted risk score and a risk score threshold.

Example 20. The one or more non-transitory computer-readable storage media of example 19, wherein the risk score threshold is a high risk score threshold, the initial prediction-based action is an entity evaluation action, and the one or more processors are further configured to generate, using an evaluation risk adjustment model, an evaluation adjusted risk score for the entity based on the disparity adjusted risk score and an evaluation data object for the entity, wherein the evaluation data object is generated based on the entity evaluation action; initiate a subsequent prediction-based action for the entity based on a comparison between the evaluation adjusted risk score and the risk score threshold, wherein the subsequent prediction-based action is an image-based entity evaluation action; generate a phenotypic profile for the entity based on the evaluation data object and an image-based evaluation data object for the entity, wherein the image-based evaluation data object is generated based on the image-based entity evaluation action; and generate a prediction-based action sequence for the entity based on the phenotypic profile.

1. A computer-implemented method, the computer-implemented method comprising:
generating, by one or more processors and using a risk prediction model, an individual risk score for an entity of a disparity group associated with an entity cohort;
generating, by the one or more processors and using a disparity risk adjustment model, a disparity adjusted risk score for the entity based on the individual risk score; and
initiating, by the one or more processors, an initial prediction-based action for the entity based on a comparison between the disparity adjusted risk score and a risk score threshold.

2. The computer-implemented method of claim 1, wherein the risk score threshold is a high risk score threshold, the initial prediction-based action is an entity evaluation action, and the computer-implemented method further comprises:
generating, using an evaluation risk adjustment model, an evaluation adjusted risk score for the entity based on the disparity adjusted risk score and an evaluation data object for the entity, wherein the evaluation data object is generated based on the entity evaluation action; and
initiating, by the one or more processors, a subsequent prediction-based action for the entity based on a comparison between the evaluation adjusted risk score and the risk score threshold.

3. The computer-implemented method of claim 2, wherein the entity evaluation action is associated with a cognitive evaluation for the entity, and wherein the cognitive evaluation is associated with at least one of a text-based entity evaluation, a speech-based entity evaluation, a gait-based entity evaluation, or a biomarker-based evaluation.

4. The computer-implemented method of claim 2, wherein the computer-implemented method further comprises:
generating, by the one or more processors and using the risk prediction model, correspondence associated with the entity evaluation action; and
providing, by the one or more processors, data indicative of the correspondence associated with the entity evaluation action.

5. The computer-implemented method of claim 2, wherein the subsequent prediction-based action is an image-based entity evaluation action, and the computer-implemented method further comprises:
generating, by the one or more processors, a phenotypic profile for the entity based on the evaluation data object and an image-based evaluation data object for the entity, wherein the image-based evaluation data object is generated based on the image-based entity evaluation action; and
generating, by the one or more processors, a prediction-based action sequence for the entity based on the phenotypic profile.

6. The computer-implemented method of claim 5, wherein the generating the phenotypic profile further comprises:
generating, by the one or more processors and using a multi-dimensional location ranking model, an entity rank associated with the entity based on the evaluation data object and the image-based evaluation data object; and
mapping, by the one or more processors, the entity rank in a multi-dimensional disease space associated with a plurality of phenotypic profiles.

7. The computer-implemented method of claim 5, wherein the phenotypic profile associated with the entity comprises at least one of a disease severity or a disease subtype and the phenotypic profile describes whether a current condition of the entity is a normal condition, reversible condition, or irreversible condition.

8. The computer-implemented method of claim 5, wherein the computer-implemented method further comprises:
generating, by the one or more processors and using the risk prediction model, a specialist referral associated with a respective medical specialist based on the prediction-based action sequence; and
providing, by the one or more processors, data indicative of the specialist referral to the entity.

9. The computer-implemented method of claim 1, wherein generating the disparity adjusted risk score further comprises:
performing, by the one or more processors, a disparity risk adjustment, wherein the disparity risk adjustment comprises:
generating, using the risk prediction model, a risk prevalence ratio, wherein the risk prevalence ratio is a ratio of a documented risk prevalence associated with the disparity group to an estimated risk prevalence associated with the disparity group,
wherein the estimated risk prevalence is determined based on aggregating the individual risk score for one or more respective entities in the disparity group associated with the entity cohort; and
generating the disparity adjusted risk score based on applying at least the risk prevalence ratio and an entity-defined disparity weighting parameter to the individual risk score associated with the entity.

10. The computer-implemented method of claim 9, wherein the estimated risk prevalence associated with the disparity group is updated based on the disparity adjusted risk score associated with the entity.

11. The computer-implemented method of claim 1, wherein the disparity group is associated with a geographic region and the disparity group is associated with one or more contextual attributes.

12. The computer-implemented method of claim 1, wherein the risk score threshold is a medium risk score threshold and the initial prediction-based action is an entity monitoring action.

13. The computer-implemented method of claim 12, wherein the computer-implemented method further comprises:
initiating, by the one or more processors based on the entity monitoring action, association of an entity monitoring computing device to the entity.

14. The computer-implemented method of claim 13, wherein the computer-implemented method further comprises:
receiving, by the one or more processors, entity monitoring data associated with the entity, wherein the entity monitoring data is generated by the entity monitoring computing device; and
updating, by the one or more processors, the individual risk score associated with the entity based on the entity monitoring data.

15. The computer-implemented method of claim 1, wherein the risk score threshold is a low risk score threshold and the initial prediction-based action is an entity evaluation cessation action.

16. A computing system comprising memory and one or more processors communicatively coupled to the memory, the one or more processors configured to:
generate, using a risk prediction model, an individual risk score for an entity of a disparity group associated with an entity cohort;
generate, using a disparity risk adjustment model, a disparity adjusted risk score for the entity based on the individual risk score; and
initiate an initial prediction-based action for the entity based on a comparison between the disparity adjusted risk score and a risk score threshold.

17. The computing system of claim 16, wherein the risk score threshold is a high risk score threshold, the initial prediction-based action is an entity evaluation action, and the one or more processors are further configured to:
generate, using an evaluation risk adjustment model, an evaluation adjusted risk score for the entity based on the disparity adjusted risk score and an evaluation data object for the entity, wherein the evaluation data object is generated based on the entity evaluation action; and
initiate a subsequent prediction-based action for the entity based on a comparison between the evaluation adjusted risk score and the risk score threshold.

18. The computing system of claim 17, wherein the subsequent prediction-based action is an image-based entity evaluation action and the and the one or more processors are further configured to:
generate a phenotypic profile for the entity based on the evaluation data object and an image-based evaluation data object for the entity, wherein the image-based evaluation data object is generated based on the image-based entity evaluation action; and
generate a prediction-based action sequence for the entity based on the phenotypic profile.

19. One or more non-transitory computer-readable storage media including instructions that, when executed by one or more processors, cause the one or more processors to:
generate, using a risk prediction model, an individual risk score for an entity of a disparity group associated with an entity cohort;
generate, using a disparity risk adjustment model, a disparity adjusted risk score for the entity based on the individual risk score; and
initiate an initial prediction-based action for the entity based on a comparison between the disparity adjusted risk score and a risk score threshold.

20. The one or more non-transitory computer-readable storage media of claim 19, wherein the risk score threshold is a high risk score threshold, the initial prediction-based action is an entity evaluation action, and the one or more processors are further configured to:
generate, using an evaluation risk adjustment model, an evaluation adjusted risk score for the entity based on the disparity adjusted risk score and an evaluation data object for the entity, wherein the evaluation data object is generated based on the entity evaluation action;
initiate a subsequent prediction-based action for the entity based on a comparison between the evaluation adjusted risk score and the risk score threshold, wherein the subsequent prediction-based action is an image-based entity evaluation action;
generate a phenotypic profile for the entity based on the evaluation data object and an image-based evaluation data object for the entity, wherein the image-based evaluation data object is generated based on the image-based entity evaluation action; and
generate a prediction-based action sequence for the entity based on the phenotypic profile.
